(19) United States
(12) Patent Application Publication
(10) Pub. No.: US 2005/0143801 A1
(43) Pub. Date: Jun. 30, 2005

(54) Systems and methods for overcoming or preventing vascular flow restrictions

(76) Inventor: Walid Najib Aboul-Hosn, Fair Oaks, CA (US)

Correspondence Address: Jonathan D. Spangler, 1780 Kettner Blvd., Unit 306, San Diego, CA, 92101, US

(21) Appl. No.: 10/840,195

(22) Filed: May 5, 2004

Related U.S. Application Data
(63) Continuation of application No. PCT/US02/32016, filed on Oct. 5, 2002.

(51) Int. Cl. A61F 2/06

(52) U.S. Cl. 623/1.11

(57) ABSTRACT

Systems and methods for overcoming or preventing vascular flow restrictions which involve: (1) providing at least one structural element within or about a vessel having a vascular flow restriction; and (2) equipping the structural element with bio-lining such that it restores blood flow and minimizes, if not eliminates, the interface between blood and non-biological materials to thereby prevent stenosis and/or restenosis.

### CROSS REFERENCE TO RELATED APPLICATIONS

This application is a continuation of PCT Patent Application Serial No. PCT/US02/32016, filed Oct. 5, 2002 and published on Apr. 17, 2003 as WO 03/030964 A2 which is incorporated herein by reference.

### BACKGROUND OF THE INVENTION

I. Field of the Invention

This invention generally relates to overcoming or preventing vascular flow restrictions for improved blood flow. More specifically, this invention relates to systems and methods which involve: (1) providing at least one structural element within or about a vessel having a vascular flow restriction; and (2) equipping the structural element with bio-lining such that it restores blood flow and minimizes, if not eliminates, the interface between blood and non-biological materials to thereby prevent restenosis.

II. Discussion of the Prior Art

Vascular stenosis is a major problem in health care worldwide, and is characterized as the narrowing (and potential blocking) of blood vessels as a result of the deposition of fatty materials, cellular debris, calcium, and/or blood clots (collectively referred to as “vascular flow restrictions”). Current treatments to overcome vascular flow restrictions include the administration of thombolytics (clot-dissolving drugs), interventional devices, and/or bypass surgery. As will be demonstrated below, these state-of-the-art techniques and devices all fail to adequately answer the vexing problem of maintaining blood flow through blood vessels.

Thrombolytics are typically administered in high doses. However, even with aggressive therapy, thrombolytics fail to restore blood flow in the affected vessel in about 30% of patients. In addition, these drugs can also dissolve beneficial clots or injure healthy tissue causing potentially fatal bleeding complications.

Interventional procedures include angioplasty, atherectomy, and laser ablation. However, the use of such devices to remove flow-restricting deposits may leave behind a wound that heals by forming a scar. The scar itself may eventually become a serious obstruction in the blood vessel (a process known as restenosis). Also, diseased blood vessels being treated with interventional devices sometimes develop vasoconstriction (elastic recoil), a process by which spasms or abrupt reclosures of the vessel occur, thereby restricting the flow of blood and necessitating further intervention. Approximately 40% of treated patients require additional treatment for restenosis resulting from scar formation occurring over a relatively long period, typically 4 to 12 months, while approximately 1-in-20 patients require treatment for vasoconstriction, which typically occurs from 4 to 72 hours after the initial treatment.

Percutaneous transluminal coronary angioplasty (PTCA), also known as balloon angioplasty, is a treatment for coronary vessel stenosis. In typical PTCA procedures, a guiding catheter is percutaneously introduced into the cardiovascular system of a patient and advanced through the aorta until the distal end is in the ostium of the desired coronary artery. Using fluoroscopy, a guide wire is then advanced through the guiding catheter and across the site to be treated in the coronary artery. A balloon catheter is advanced over the guide wire to the treatment site. The balloon is then expanded to reopen the artery. The increasing popularity of the PTCA procedure is attributable to its relatively high success rate, and its minimal invasiveness compared with coronary by-pass surgery.

The benefit of balloon angioplasty, especially of the coronary arteries, has been amply demonstrated over the past decade. Angioplasty is effective to open occluded vessels that would, if left untreated, result in myocardial infarction or other cardiac disease or dysfunction. These benefits are diminished, however, by restenosis rates approaching 50% of the patient population that undergo the procedure. Restenosis is believed to be a natural healing reaction to the injury of the arterial wall that is caused by angioplasty procedures. The healing reaction begins with the clotting of blood at the site of the injury. The final result of the complex steps of the healing process is intimal hyperplasia, the migration and proliferation of medial smooth muscle cells (in a mechanism analogous to wound healing and scar tissue), until the artery is again stenotic or occluded. Such reocclusion may even exceed the clogging that prompted resort to the original angioplasty procedure. Accordingly, a huge number of patients experiencing a successful primary percutaneous transluminal coronary angioplasty (PTCA) procedure are destined to require a repeat procedure. The patient faces an impact on his or her tolerance and well being, as well as the considerable cost associated with repeat angioplasty.

To reduce the likelihood of reclosure of the vessel, it has become common practice for the physician to implant a stent in the patient at the site of the angioplasty or artherectomy procedure, immediately following that procedure, as a prophylactic measure. A stent is typically composed of a biologically compatible material (biomaterial) such as a biocompatible metal wire of tubular shape or metallic perforated tube. The stent should be of sufficient strength and rigidity to maintain its shape after deployment, and to resist the elastic recoil of the artery that occurs after the vessel wall has been stretched. The deployment procedure involves advancing the stent on a balloon catheter to the designated site of the prior (or even contemporaneous) procedure under fluoroscopic observation. When the stent is positioned at the proper site, the balloon is inflated to expand the stent radially to a diameter at or slightly larger than the normal unobstructed inner diameter of the arterial wall, for permanent retention at the site. The stent implant procedure from the time of initial insertion to the time of retracting the balloon is relatively brief, and certainly far less invasive than coronary bypass surgery. In this fashion, the use of stents has constituted a beacon in avoidance of the complication, risks, potential myocardial infarction, need for emergency bypass operation, and repeat angioplasty that would be present without the stenting procedure.

Despite its considerable benefits, coronary stenting alone is not a panacea, as studies have shown that about 30% of the patient population subjected to that procedure will still experience restenosis (referred to hereinafter as “in-stent restenosis”). While this percentage is still quite favorable compared to the approximate 50% recurrence rate for patients who have had a PTCA procedure without stent insertion at the angioplasty site, improvement is nonetheless needed to reduce the incidence of in-stent restenosis. In the past few years, considerable research has been devoted worldwide to studying the mechanisms of in-stent restenosis. It has been shown that the very presence of the stent in the blood stream may induce a local or even systemic activation of the patient's hemostase coagulation system, resulting in local thrombus formation which, over time, may restrict the flow of blood.

To avoid this problem, various efforts have been undertaken to coat or treat the surface of the stent to prevent or minimize thrombus formation. One approach to reducing in-stent restenosis involves coating the stent with a biocompatible, non-foreign body-inducing, biodegradable polylactic acid of thin paint-like thickness in a range below 100 microns, and preferably about 10 microns thick. Animal research has shown that a 30% reduction in in-stent restenosis may be achieved using this technique. This thin coating on a metallic stent may be used to release drugs incorporated therein, such as hirudin and/or a platelet inhibitor such as prostacyclin (PGI.sub.2), a prostaglandin. Both of these drugs are effective to inhibit proliferation of smooth muscle cells, and decrease the activation of the intrinsic and extrinsic coagulation system. Therefore, the potential for a very significant reduction in restenosis has been demonstrated in these animal experiments.

Other coating techniques involve coating the stent with a biodegradable substance or composition which undergoes continuous degradation in the presence of body fluids such as blood, to self-cleanse the surface as well as to release thrombus inhibitors incorporated in the coating. Disintegration of the carrier occurs slowly through hydrolytic, enzymatic or other degenerative processes. The biodegradable coating acts to prevent the adhesion of thrombi to the biomaterial or the coating surface, especially as a result of the inhibitors in the coating, which undergo slow release with the controlled degradation of the carrier. Blood components such as albumin, adhesive proteins, and thrombocytes can adhere to the surface of the biomaterial, if at all, for only very limited time because of the continuous cleansing action along the entire surface that results from the ongoing biodegradation.

Materials used for the biodegradable coating and the slow, continuous release of drugs incorporated therein include synthetic and naturally occurring aliphatic and hydroxy polymers of lactic acid, glycolic acid, mixed polymers and blends. Alternative materials for those purposes include biodegradable synthetic polymers such as polyhydroxybutyrates, polyhydroxyvaleriates and blends, and polydioxanon, modified starch, gelatine, modified cellulose, caprolactaine polymers, acrylic acid and methacrylic acid and their derivatives. It is important that the coating have tight adhesion to the surface of the biomaterial, which can be accomplished by applying the aforementioned thin, paint-like coating of the biodegradable material that may have coagulation inhibitors blended therein, as by dipping or spraying, followed by drying, before implanting the coated biomaterial device.

Anti-proliferation substances may be incorporated into the coating carrier to slow proliferation of smooth muscle cells at the internal surface of the vascular wall. Such substances include corticoids and dexamethasone, which prevent local inflammation and further inducement of clotting by mediators of inflammation. Substances such as taxol, tamoxifen and other cytostatic drugs directly interfere with intimal and medial hyperplasia, to slow or prevent restenosis, especially when incorporated into the coating carrier for slow release during biodegradation. Local relaxation of a vessel can be achieved by inclusion of nitrogen monoxide (NO) or other drugs that release NO, such as organic nitrates or molsidomin, or SIN1, its biologically effective metabolite.

The amount and dosage of the drug or combination of drugs incorporated into and released from the biodegradable carrier material is adjusted to produce a local suppression of the thrombotic and restenotic processes, while allowing systemic clotting of the blood. The active period of the coated stent may be adjusted by varying the thickness of the coating, the specific type of biodegradable material selected for the carrier, and the specific time release of incorporated drugs or other substances selected to prevent thrombus formation or attachment, subsequent restenosis and inflammation of the vessel.

The biodegradable coating may also be applied to the stent in multiple layers, either to achieve a desired thickness of the overall coating or a portion thereof for prolonged action, or to employ a different beneficial substance or substances in each layer to provide a desired response during a particular period following implantation of the coated stent. For example, at the moment the stent is introduced into the vessel, thrombus formation will commence, so that a need exists for a top layer if not the entire layer of the coating to be most effective against this early thrombus formation, with a relatively rapid release of the incorporated, potent anticoagulation drug to complement the self-cleansing action of the disintegrating carrier. For the longer term of two weeks to three months after implantation, greater concern resides in the possibility of intimal hyperplasia that can again narrow or fully obstruct the lumen of the vessel. Hence, the same substance as was present or a different substance from that in the top layer might be selected for use in the application of the coating to meet such exigencies. Hirudin, for example, can be effective against both of these mechanisms or phenomena.

A still further technique for preventing restenosis involves the use of radiation. U.S. Pat. No. 4,768,507 to Fischell et al. proposes in the use of a special percutaneous insertion catheter for purposes of enhancing luminal dilatation, preventing arterial restenosis, and preventing vessel blockage resulting from intimal dissection following balloon and other methods of angioplasty. U.S. Pat. No. 4,779,641 and co-pending European patent application No. 92309580.6 disclose the use of an interbiliary duct stent, wherein radioactive coils of a wire are embedded into the interior wall of the bile duct to prevent restenotic processes from occurring. U.S. Pat. No. 4,448,691 and co-pending European patent application No. 90313433.6 disclose a helical wire stent, provided for insertion into an artery following balloon angioplasty or atherectomy, which incorporates or is plated with a radioisotope to decrease the proliferation of smooth muscle cells. The disclosure teaches that the stent may be made radioactive by irradiation or by incorporating a radioisotope into the material of which the stent is composed. Another solution would be to locate the radioisotope at the core of the tubular stent or to plate the radioisotope onto the surface of the stent. The patent also teaches, aside from the provision of radioactivity of the stent, that an outer coating of anti-thrombogenic material might be applied to the stent.

U.S. Pat. No. 5,059,166 to Fischell et al. discloses a helical coil spring stent composed of a pure metal which is made radioactive by irradiation. Alternative embodiments disclosed in summary fashion in the patent include a steel helical stent which is alloyed with a metal that can be made radioactive, such as phosphorus (14.3 day half life); or a helical coil which has a radioisotope core and a spring material covering over the core; or a coil spring core plated with a radioisotope such as gold 198 (Au.sup.198, which has a half life of 2.7 days), which may be coated with an anti-thrombogenic layer of carbon.

Clinical basic science reports such as “Inhibition of neointimal proliferation with low dose irradiation from a beta particle emitting stent” by John Laird et al published in Circulation (93: 529-536, 1996) describe creating a beta particle-emitting stent by bombarding the outside of a titanium wire with phosphorus. The implantation of phosphorus into the titanium wire was achieved by placing the P.sup.31 into a special vacuum apparatus, and then vaporizing, ionizing and, accelerating the ions with a higher voltage so that the P.sup.31 atoms become buried beneath the surface of the titanium wire in a thickness of about ⅓ micron. After exposing the wire together with the phosphorus radioisotope for several hours to a flux of slow neutrons part of the P.sup.31 atoms were converted into a P.sup.32, a pure beta particle emitter with a maximum energy of 1.709 megaelectron-volts, an average of 0.695 megaelectron-volts, and a half-life of 14.6 days.

Despite the convincing clinical results obtained by this method, practical application of the method in human patients raises considerable concerns. First, it is difficult to create a pure beta emitter from phosphorus if a stent is exposed to a flux of slow neutrons. In addition to converting phosphorus from P.sup.31 to P.sup.32, the metallic structure of the titanium wire will become radioactive. Therefore, about 20 days are needed to allow the radiation to decay, especially gamma radiation which originates from the titanium wire. Even worse is the situation where a metal such as stainless steel undergoes radioactive irradiation, resulting in production of unwanted .gamma. radiation and a wide range of short and long term radionuclei such as cobalt.sup.57, iron.sup.55, zinc.sup.65, molybdenum.sup.99, cobalt.sup.55. A pure beta radiation emitter with a penetration depth of about 3 millimeters is clearly superior for a radioactive stent for purposes of local action, side effects, and handling.

Reports have indicated that good results have been obtained with a radioactive wire inserted into the coronary arteries or into arteriosclerotic vessels of animals. Results obtained with a gamma radiation source from a wire stems from the deeper penetration of gamma radiation, which is about 10 mm. Assuming that the vessel is 3 to 4 mm in diameter, a distance of 2 to 4 mm depending on the actual placement of the wire toward a side wall has to be overcome before the radiation acts. Therefore, the clinical results that have been obtained with radioactive guide wires that have been inserted into the coronary arteries for a period ranging from about 4 to 20 minutes for delivery of a total dosage of about 8 to 18 Gray (Gy) have shown that gamma radiation has a beneficial effect while beta radiation from a wire is less favorable. On the other hand, gamma radiation which originates from a stainless steel stent such as composed of 316 L is less favorable since the properties of .beta. radiation such as a short half-life and a short penetration depth are superior to .gamma. radiation originating from radioactive 316 L with a long half-life and a deeper penetration since the proliferative processes of smooth muscle cell proliferation occur within the first 20 to 30 days and only in the very close vicinity of the stent.

In addition, a half-life which is too short such as one to two days considerably impacts on logistics if a metallic stent needs to be made radioactive. That is, by the time the stent is ready for use, its radioactivity level may have decayed to a point which makes it unsuitable for the intended purpose.

Another technique for preventing in-stent restenosis involves providing stents seeded with endothelial cells (Dichek, D. A. et al Seeding of Intravascular Stents With Genetically Engineered Endothelial Cells; Circulation 1989; 80: 1347-1353). In that experiment, sheep endothelial cells that had undergone retrovirus-mediated gene transfer for either bacterial beta-galactosidase or human tissue-type plasmogen activator were seeded onto stainless steel stents and grown until the stents were covered. The cells were therefore able to be delivered to the vascular wall where they could provide therapeutic proteins. Other methods of providing therapeutic substances to the vascular wall by means of stents have also been proposed such as in international patent application WO 91/12779 “Intraluminal Drug Eluting Prosthesis” and international patent application WO 90/13332 “Stent With Sustained Drug Delivery”. In those applications, it is suggested that antiplatelet agents, anticoagulant agents, antimicrobial agents, antimetabolic agents and other drugs could be supplied in stents to reduce the incidence of restenosis.

In the vascular graft art, it has been noted that fibrin can be used to produce a biocompatible surface. For example, in an article by Soldani et al., “Bioartificial Polymeric Materials Obtained from Blends of Synthetic Polymers with Fibrin and Collagen” International Journal of Artificial Organs, Vol. 14, No. 5, 1991, polyurethane is combined with fibrinogen and cross-linked with thrombin and then made into vascular grafts. In vivo tests of the vascular grafts reported in the article indicated that the fibrin facilitated tissue ingrowth and was rapidly degraded and reabsorbed. Also, in published European Patent Application 0366564 applied for by Terumo Kabushiki Kaisha, Tokyo, Japan, discloses a medical device such as an artificial blood vessel, catheter or artificial internal organ is made from a polymerized protein such as fibrin. The fibrin is said to be highly nonthrombogenic and tissue compatible and promotes the uniform propagation of cells that regenerates the intima. Also, in an article by Gusti et al., “New Biolized Polymers for Cardiovascular Applications”, Life Support Systems, Vol. 3, Suppl. 1, 1986, “biolized” polymers were made by mixing synthetic polymers with fibrinogen and cross-linking them with thrombin to improve tissue ingrowth and neointima formation as the fibrin biodegrades. Also, in an article by Haverich et al., “Evaluation of Fibrin Seal in Animal Experiments”, Thoracic Cardiovascular Surgeon, Vol. 30, No. 4, pp. 215-22, 1982, the authors report the successful sealing of vascular grafts with fibrin. However, none of these teach that the problem of restenosis could be addressed by the use of fibrin and, in fact, conventional treatment with anticoagulant drugs following angioplasty procedures is undertaken because the formation of blood clots (which include fibrin) at the site of treatment is thought to be undesirable.

As evidenced by the foregoing, the prior art is replete with attempts at solving the problem of vascular flow restrictions. Notwithstanding these efforts, the prior art systems and methods all suffer significant drawbacks which inhibit widespread adoption and success, as evidenced by the multitude of attempts in this area. The present invention is directed at overcoming, or at least reducing the effects of, one or more of the problems set forth above.

### SUMMARY OF THE INVENTION

The present invention helps overcome the drawbacks of the prior art by providing systems and methods for overcoming or preventing vascular flow restrictions. More specifically, the present invention includes systems and methods which involve solve the problems in the prior art by: (1) providing at least one structural element within or about a vessel having a vascular flow restriction; and (2) equipping the structural element with bio-lining such that it restores blood flow and minimizes, if not eliminates, the interface between blood and non-biological materials. By reducing or eliminating this “blood-device” interface, the present invention prevents (or at the very least lessens) the re-formation of vascular flow restrictions within the diseased vessel (otherwise known as “vascular restenosis”). The various systems and methods described below all address the goal of overcoming vascular flow restrictions for improved blood flow.

In one broad aspect, the present invention overcomes or prevents vascular flow restrictions by providing a bio-lined structural element for placement within a diseased or occluded blood vessel. The structural element may comprise any number of devices or components capable of providing sufficient structural support to maintain the lumen of a blood vessel in a sufficiently open and unrestricted state once deployed within or about the blood vessel. Such devices or components may include, but are not necessarily limited to, any number of stent or stent-like devices of generally tubular, meshed construction. The bio-lining provided within the structural element may comprise any number of lining materials having characteristics which prevent or reduce the formation of vascular flow restrictions when deployed within a blood vessel. Such lining materials may include, but are not necessarily limited to, autologous vessel (harvested from the patient), tissue-engineered vessel (preferably based on the patient's own DNA), or synthetic vessel, or combination of any or all above-mentioned tissue.

Still other broad aspects of the present invention involve preparing the bio-lined structural element for use in overcoming vascular flow restrictions. One such aspect involves harvesting autologous tissue from the patient for use as the bio-lining according to the present invention. A more particular aspect involves implanting the structural element over a blood vessel within the patient for a sufficient duration such that the blood vessel actually grows into (and becomes imbedded within) the structural element and can be thereafter harvested and used in the patient. A still further aspect involves harvesting a length of autologous blood vessel for immediate affixation within the structural element, such as through the use of cutting devices and/or cutting catheters. Yet another aspect involves equipping a structural element with a bio-lining created through tissue-engineering techniques.

Further broad aspects of the present invention involve overcoming vascular flow restrictions by disposing a structural element about some or all of the periphery of a native vessel suffering from a vascular flow restriction and thereafter affixing the structural element to the native vessel. By buttressing the vessel in this fashion, the lumen of the vessel suffering the vascular flow restriction may become “opened” or otherwise widened to increase the inner diameter, thereby producing improved blood flow.

Still other broad aspects of the present invention involve overcoming vascular flow restrictions by providing a pair of bio-lined structural elements disposed a distance from one another and connected by a length of bio-lining. In this fashion, each of the bio-lined structural elements may be deployed on either side of a vascular flow restriction such that flow is restored through the length of bio-lining that extends there between.

Other features and advantages of the invention will become apparent from the following detailed description, taken in conjunction with the accompanying drawings, which illustrate, by way of example, the features of the invention.

### BRIEF DESCRIPTION OF THE DRAWINGS

The following description of the preferred embodiments of the present invention will be better understood in conjunction with the appended drawings, in which:

FIG. 1 is a cross-sectional view of a bio-lined structural element according to one aspect of the present invention;

FIG. 2 is a cross-sectional view of a bio-lined structural element according to one aspect of the present invention;

FIG. 3 is a cross-sectional view of a bio-lined structural element according to one aspect of the present invention;

FIG. 4 is an enlarged view of a coupling member according to one aspect of the present invention;

FIG. 5 is a cross-sectional view of a deployment catheter according to one aspect of the present invention;

FIG. 6 is an enlarged view of a catheter body of the deployment catheter shown in FIG. 5;

FIG. 7 is a cross-sectional view of the catheter body taken through lines 7-7 in FIG. 6;

FIG. 8 is an enlarged view of a coupling member disposed within the wall of the balloon of the deployment catheter of FIG. 5;

FIG. 9 is an enlarged view showing a plurality of alternate coupling members for use in the present invention;

FIG. 10 is a cross-sectional view of a bio-lined structural element employing an inner structural element according to one aspect of the present invention;

FIG. 11 is a side view of an inner structural according to one aspect of the present invention;

FIG. 12 is a top view of an inner structural element according to one aspect of the present invention;

FIG. 13 is a side view of an inner structural element according to one aspect of the present invention;

FIG. 14 is a side view of an inner structural element according to one aspect of the present invention;

FIG. 15 is a side view of the inner structural element shown in FIG. 14;

FIG. 16 is a cross-sectional view illustrating the method of implanting a structural element over a length of autologous blood vessel to prepare a bio-lined structural element according to the present invention;

FIG. 17 is a perspective view of a structural element for implanting over a length of autologous blood vessel to produce a bio-lined structural element according to one aspect of the present invention;

FIG. 18 is a perspective view of a structural element for implanting over a length of autologous blood vessel to produce a bio-lined structural element according to one aspect of the present invention;

FIG. 19 is a cross-sectional view of a structural element immediately upon implantation according to one aspect of the present invention;

FIG. 20 is a cross-sectional view of a structural element after a period of implantation according to one aspect of the present invention;

FIG. 21 is a cross-sectional view illustrating a step of harvesting the bio-lined structural element according to one aspect of the present invention;

FIG. 22 is a cross-sectional view illustrating a step of harvesting the bio-lined structural element according to one aspect of the present invention;

FIG. 23 is a cross-sectional view illustrating the method of implanting a structural element over two lengths of autologous blood vessel to prepare a bio-lined structural element according to the present invention;

FIG. 24 is a partial cross-sectional view of a cutting catheter according to one aspect of the present invention;

FIGS. 25-27 are cross-sectional views illustrating the introduction of a guidewire and preparation of a target vessel for harvesting autologous bio-lining according to one aspect of the present invention;

FIG. 28 is a partial cross-sectional view illustrating a dilator and introducer positioned within the target vessel following the steps shown in FIGS. 25-27 according to one aspect of the present invention;

FIG. 29 is a partial cross-sectional view illustrating the advancement of the cutting catheter shown in FIG. 24 over the dilator and introducer according to one aspect of the present invention;

FIG. 30 is a partial cross-sectional view illustrating the cutting catheter advanced to extricate the target autologous bio-lining from surrounding tissue and a deployment catheter of the type shown in FIGS. 5-8 disposed within the introducer according to one aspect of the present invention;

FIG. 31 is a partial cross-sectional view illustrating the deployment catheter in use (deploying coupling members into autologous bio-lining within a patient) after being advanced through the end of the introducer and past the cutting catheter according to one aspect of the present invention;

FIG. 32 is a partial cross-sectional view illustrating the cutting catheter in use after the deployment has been employed to deploy the coupling members into the autologous bio-lining according to one aspect of the present invention;

FIG. 33 is a partial cross-sectional view illustrating the step of severing the distal end of the autologous bio-lining for withdrawal from the patient according to one aspect of the present invention;

FIG. 34 is a partial cross-sectional view illustrating the step of severing the distal end of the autologous bio-lining for withdrawal from the patient according to one aspect of the present invention;

FIGS. 35-39 are partial cross-sectional views illustrating alternate embodiments of the cutting catheter according to several aspects of the present invention;

FIG. 40 is a partial cross-sectional view of a holding catheter according to one aspect of the present invention;

FIG. 41 is a partial cross-sectional view of the holding catheter shown in FIG. 40 in use according to one aspect of the present invention;

FIG. 42 is a side view of a windowed cutting catheter according to one aspect of the present invention;

FIGS. 43-44 are cross-sectional views of a bio-lined structural element according to one aspect of the present invention;

FIGS. 45-46 are cross-sectional views of a bio-lined structural element according to one aspect of the present invention;

FIGS. 47-48 are cross-sectional views of a bio-lined structural element according to one aspect of the present invention;

FIGS. 49-52 are cross-sectional views illustrating a connector assembly and its use for connecting the two lengths of bio-lining to form the bio-lined structural element shown in FIGS. 47-48 according to one aspect of the present invention;

FIGS. 53-55 are cross-sectional views illustrating a connector assembly and its use for connecting the two lengths of bio-lining to form the bio-lined structural element shown in FIGS. 47-48 according to one aspect of the present invention;

FIGS. 56-57 are cross-sectional views illustrating the manner in which structural elements of the type shown in FIGS. 47-48 are coupled to the bio-lining according to one aspect of the present invention;

FIG. 58 is a cross-sectional view illustrating the manner in which structural elements of the type shown in FIGS. 47-48 are coupled to the bio-lining according to one aspect of the present invention;

FIGS. 59-60 are cross-sectional views illustrating the manner in which structural elements of the type shown in FIGS. 47-48 are coupled to the bio-lining according to one aspect of the present invention;

FIGS. 61-62 are cross-sectional views illustrating the manner in which structural elements of the type shown in FIGS. 47-48 are coupled to the bio-lining according to one aspect of the present invention;

FIG. 63 a cross-sectional view of a bio-lined structural element according to one aspect of the present invention;

FIGS. 64-68 are side views illustrating a manner of introducing a self-expanding bio-lined structural element within a blood vessel according to one aspect of the present invention;

FIG. 69 is side view illustrating a constricting device and self-expanding structural element according to one aspect of the present invention;

FIG. 70 is an enlarged view of the self-expanding structural element according to one aspect of the present invention;

FIG. 71 is a top view of the constricting device of the type shown in FIG. 69 according to one aspect of the present invention; and

FIG. 72 is a side view of the handle member of the constriction device according to one aspect of the present invention.

### DESCRIPTION OF THE PREFERRED EMBODIMENT

Illustrative embodiments of the present invention are described below. In the interest of clarity, all features of an actual implementation may not be described in this specification. It will of course be appreciated that in the development of any such actual embodiment, numerous implementation-specific decisions must be made to achieve the developers' specific goals, such as compliance with business-related constraints, which may vary from one implementation to another. Moreover, it will be appreciated that such a development effort might be complex and time-consuming, but would nevertheless be a routine undertaking for those of ordinary skill in the art having the benefit of this disclosure.

The present invention provides systems and methods for overcoming or preventing vascular flow restrictions which involve minimizing (if not eliminating) the extent to which blood interfaces with a structural element deployed within or about a diseased vessel to restore blood flow. By reducing or eliminating this “blood-device” interface, the present invention prevents (or at the very least lessens) the re-formation of vascular flow restrictions within the diseased vessel (otherwise known as “vascular restenosis”). The various systems and methods described below all address the goal of overcoming vascular flow restrictions for improved blood flow. Although set forth individually, it will be appreciated that the various features of any given design or system disclosed herein may be combined with those of other designs or systems disclosed herein without departing from the scope of the present invention.

I. Bio-Lined Structural Element

In one broad aspect, the present invention overcomes the problems of the prior art by providing a bio-lined structural element for placement within a diseased blood vessel. The structural element may comprise any number of devices or components capable of providing sufficient structural support to maintain the lumen of a blood vessel in a sufficiently open and unrestricted state once deployed within or about the blood vessel. Such devices or components may include, but are not necessarily limited to, any number of stent or stent-like devices of generally tubular, meshed construction. The bio-lining provided within the structural element may comprise any number of lining materials having characteristics which prevent or reduce the formation of vascular flow restrictions when deployed within a blood vessel. Such lining materials may include, but are not necessarily limited to, autologous vessel (harvested from the patient), tissue-engineered vessel (preferably based on the patient's own DNA), or synthetic vessel, or combination of any or all above-mentioned tissue. The structural element and/or bio-lining may also be equipped with a therapeutic agent capable of inhibiting smooth muscle cell proliferation and/or proliferation or migration of fibroblast cells, including but not limited to a combination of therapeutic agents, such as a first agent of paclitaxel and a second therapeutic agent of camptothecin, colchicine or dexamethasone.

A. Bio-Lined Structural Element Design(s)

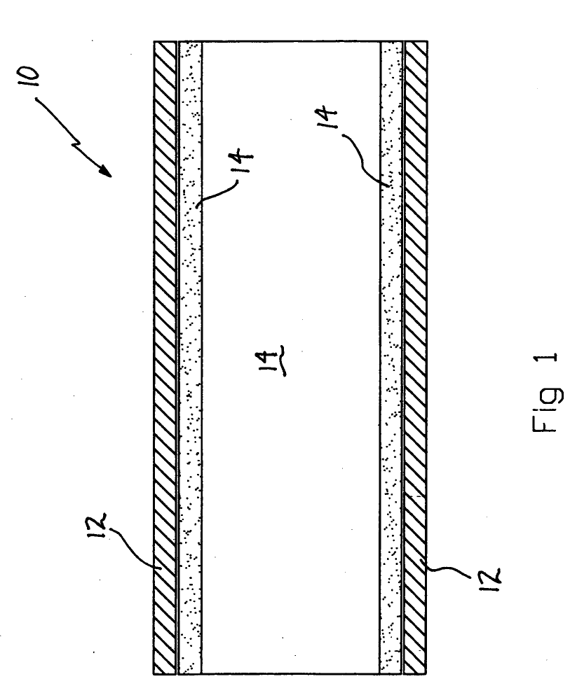

FIG. 1 illustrates a bio-lined structural element 10 according to a first broad aspect of the present invention. The bio-lined structural element 10 includes a structural element 12 having a length of bio-lining 14 disposed therein. The bio-lining 14 may be adhered, affixed, or otherwise coupled to the interior of the structural element 12 using any number of different methods, manners, compositions, or devices (several of which will be discussed below by way of example only). The structural element 12 is preferably of a radially expandable construction such that it can be introduced into a diseased or occluded blood vessel while in a contracted state of minimal diameter and thereafter deployed into an expanded state of increased diameter. Deploying the bio-lined structural element 10 is this fashion serves to maintain or restore blood flow therethrough the diseased or occluded vessel. In an important aspect, the bio-lining 14 prevents the blood from interfacing or contacting the structural element 12 or the diseased wall of the vessel (i.e. coronary artery). In this fashion, the bio-lined structural element 10 of the present invention prevents or minimizes restenosis within the diseased blood vessel.

In one embodiment, the structural element 12 comprises a stent having a generally tubular, meshed construction and the bio-lining 14 comprises a length of autologous vessel harvested from the patient. It will be appreciated, however, these choices are set forth by way of example only and are in no way limiting on the broad scope of the present invention. When provided as a stent, the structural element 12 may be of self-expanding or balloon-expandable construction. Structural element 12 may be composed of any number of different biocompatible materials, including but not limited to biocompatible metals (such as stainless steel, titanium, tungsten, tantalum, gold, platinum, cobalt, iridium, alloys thereof, and shape-memory alloys) and biocompatible polymers or plastics (such as polytetra-fluoroethylene (PTFE), polyamides, polyimides, silicones, acrylates, methacrylates, fluorinated polymers, homo-polymers, copolymers or polymer blends. By way of example only, the structural element 12 may be a stent composed of a copolymer of acrylate and methacrylate, such as that described in U.S. Pat. No. 5,163,952 (the contents of which is incorporated by reference in its entirety).

The structural element 12 and bio-lining 14 may have a selected axial length and maximum diameter determined according to the size of the lesion or treatment area within the blood vessel and the diameter of the blood vessel. Although not shown, the bio-lining 14 may be sized slightly longer than the structural element 12 in order to fold or dispose the ends of the bio-lining 14 over the ends of the structural element 12. This effectively covers the ends of the structural element 12 to further reduce the blood-device interface once deployed within a treatment site. Although not shown, the structural element 12 may also be equipped with an outer sleeve or element (of biocompatible polymeric and/or metallic construction) capable of being positioned over the structural element 12. Such an outer sleeve or element may be useful in bolstering the strength of the structural element 12, covering any sharp edges on the structural element 12, and/or preventing the protrusion of any diseased vessel through the structural element 12 that may otherwise contact the exterior surface of the bio-lining and possibly affect the form or function of the bio-lined structural element.

Any of a variety of techniques may be employed to affix or otherwise couple the bio-lining 14 within the structural element 12 according to the present invention, including mechanical or adhesive technology. Such mechanical coupling may be accomplished, for example, via barbed coupling members, ultrasonic welding, resistive heating and laser irradiation. Such adhesive coupling may be accomplished, for example, via fluorinated thermoplastic polymer adhesives such as fluorinated ethylene/propylene copolymers, perfluoroalkoxy fluoro-carbons, ethylene/tetrafluoroethylene copolymers, fluoroacrylates, and fluorinated polyvinyl ethers.

Still other techniques involve the use of bio-compatible adhesives. That is, any of a variety of suitable bio-compatible adhesives (including but not limited to UV-activated bio-glue and/or fibrin) may be employed to affix the bio-lining 14 within the structural element 12. This can be accomplished (by way of example only) via the following method: (a) applying adhesive to the exterior of the bio-lining 14 and/or interior surface of the structural element 12; (b) advancing the bio-lining 14 into the structural element 12; (c) bringing the exterior surface of the bio-lining 14 into contact with the interior surface of the structural element 12; and (d) curing the glue. In one embodiment, step (c) may be accomplished by introducing an instrument through the lumen of the bio-lining 14, wherein the instrument is dimensioned to expand the bio-lining 14 such that it is brought into abutting relation with the interior surface of the structural element 12. When employing UV-activated bio-glue, step (d) may be accomplished by subjecting the bio-lining 14 and structural element 12 to ultra-violet light in an amount and/or duration sufficient to cure the bio-glue.

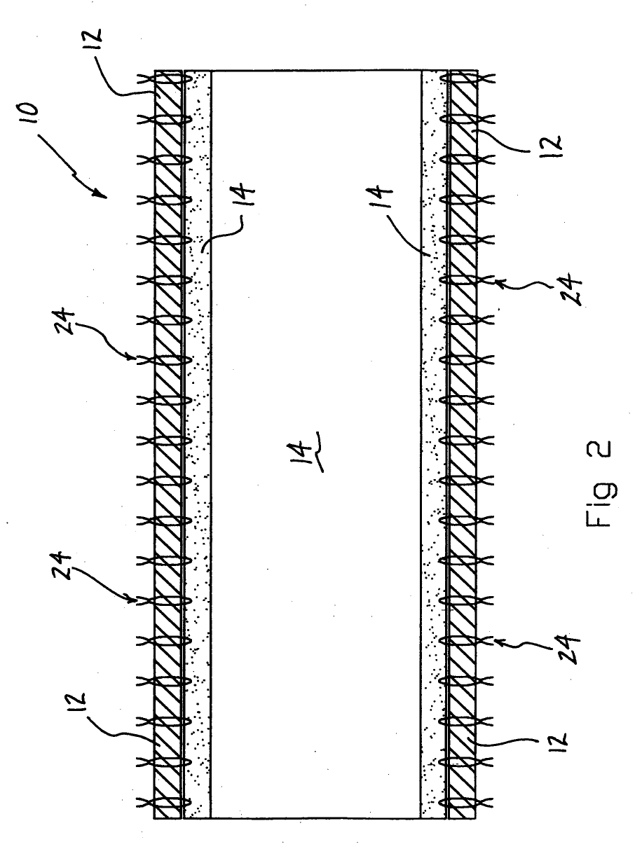

FIG. 2 illustrates, by way of example only, one manner of coupling the bio-lining 14 to the structural element 12. Namely, a plurality of sutures 24 may be provided to physically couple or attach the bio-lining 14 to the interior of the structural element 12. To create the sutures 24, a surgeon may simply advance a needle (not shown) through the structural element 12 such that the suture material extends into the bio-lining 14 and returns through the structural element 12 to be tied off. By creating a plurality of such sutures 24, the bio-lining 14 will be effectively coupled to the interior of the structural element 12 such that the combination may thereafter be contracted, introduced into a treatment site, and deployed for improved blood flow according to the present invention.

The sutures 24 may comprise any number of bio-compatible suture or devices which perform suture-like functions, including but not limited to sutures, thread-like materials, and/or surgical staples. Sutures 24 may also comprise any of a variety of bio-degradable materials, including but not limited to fibrin and/or collagen-based materials. In this fashion, the sutures 24 will be able to maintain the bio-lining 14 securely within the structural element 12 for a sufficient period to promote the ingrowth of the bio-lining 14 into the structural element 12. At some point after such ingrowth, the sutures 24 will deteriorate according to their bio-degradable characteristics, thereby removing any “dimples” on the interior of the bio-lining 14 that may sometimes occur due to the sutures 24. Removal of such “dimples” advantageously makes the interior of the bio-lining 14 as smooth as possible for improved laminar blood flow past the treatment site.

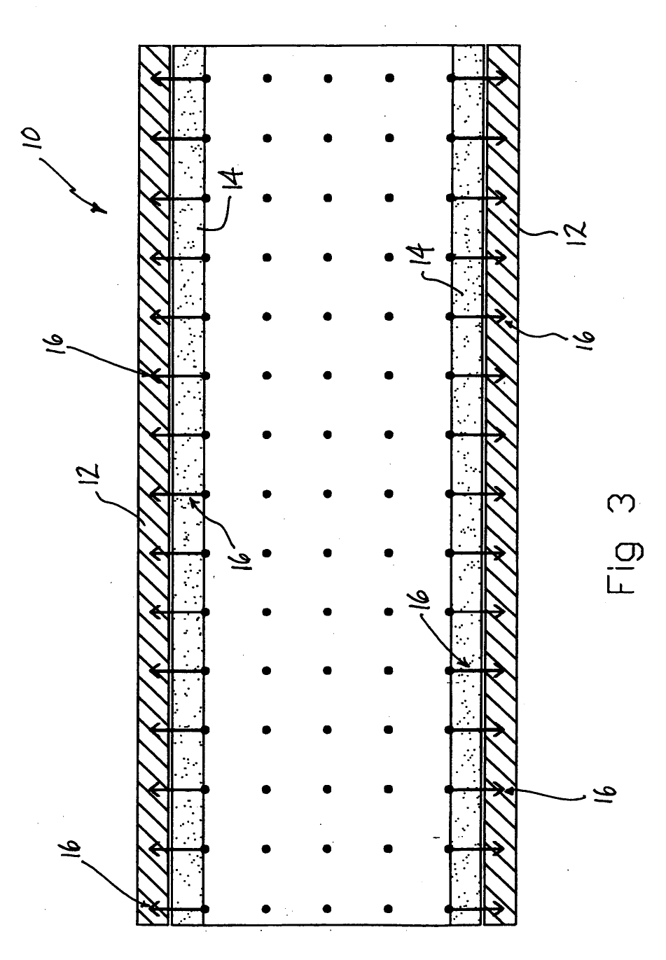

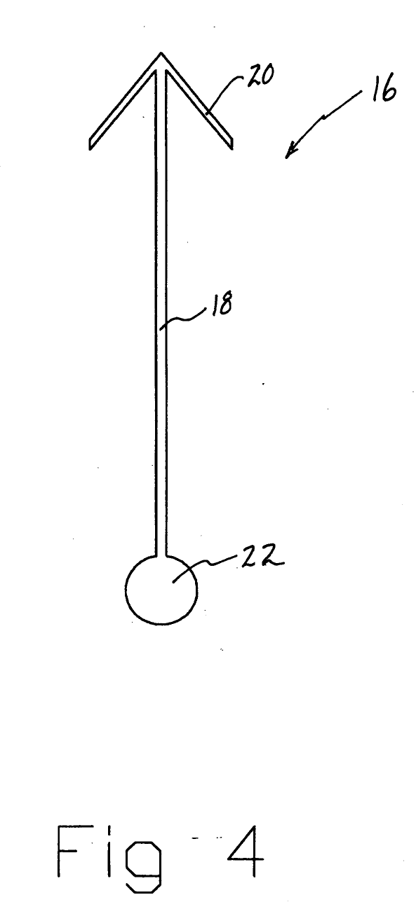

FIG. 3 illustrates, by way of example only, yet another manner of coupling the bio-lining 14 to the structural element 12. Namely, a plurality of coupling members 16 are provided to physically couple or attach the bio-lining 14 to the interior of the structural element 12. With combined reference to FIGS. 3 and 4, each coupling member 16 includes a shaft 18 having a penetrating tip 20 at the distal end and an enlarged base 22 at the proximal end. As will be discussed in greater detail below, the coupling members 16 are designed such that, upon deployment, the penetrating tips 20 pass through the bio-lining 14 and engage with the structural element 12, while the enlarged bases 22 abut against the interior of the bio-lining 14. In this fashion, deployment of the coupling members 16 draws the bio-lining 14 into contact with the interior of the structural element 12 and thereby affixes the bio-lining 14 within the structural element 12.

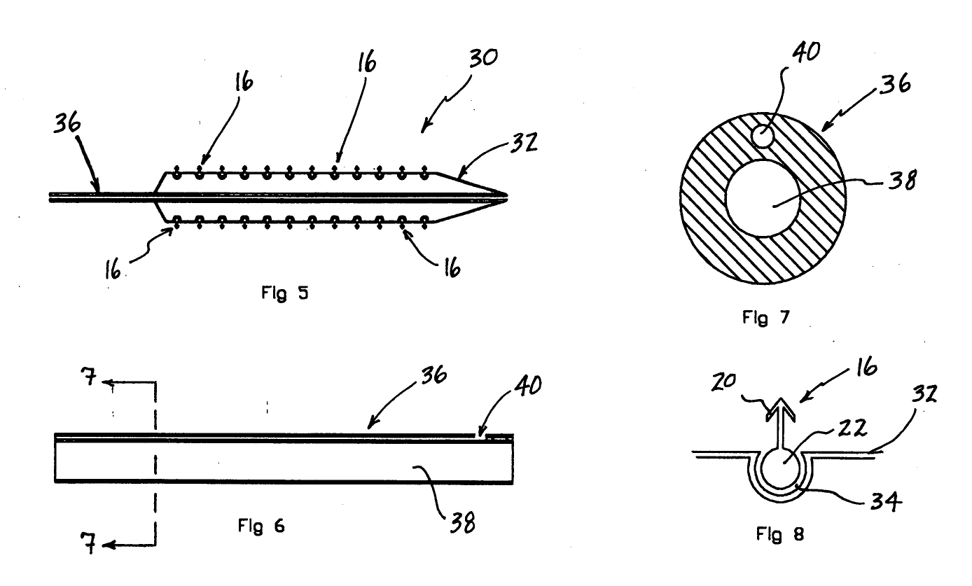

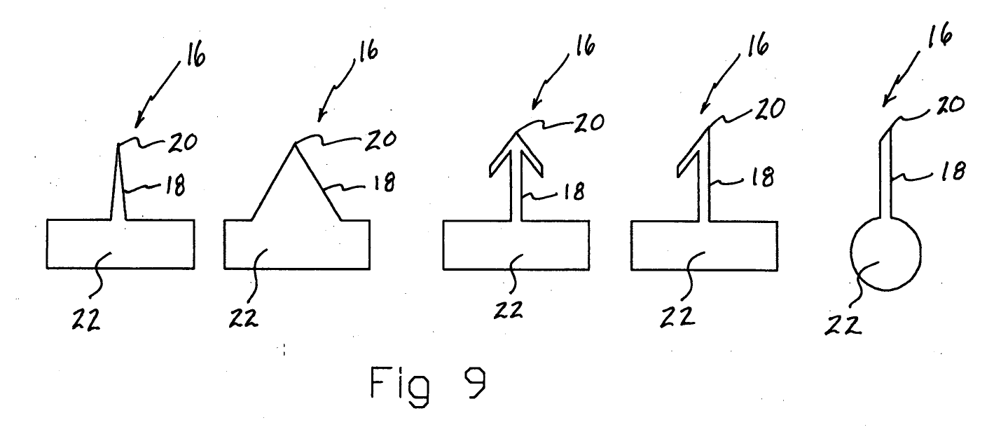

FIGS. 5-8 illustrate one manner of deploying the coupling members 16 according to the present invention. Referring initially to FIG. 5, a deployment catheter 30 is provided having a catheter body 36 with an inflatable balloon 32 disposed on the distal end thereof. With reference to FIGS. 5-7, the catheter body 36 is preferably of multi-lumen construction, having a centrally located guide-wire lumen 38 and an inflation lumen 40. Referring to FIGS. 5 and 8, the balloon 32 is designed to receive a plurality of coupling members 16. This may be accomplished, for example, by encapsulating the coupling members 16 (at least partially) within the wall of the balloon 32 during manufacture of the balloon 32 (such as through injection molding). Another possible fabrication method entails dipping in urethane or silicone a cylinder that is holding several coupling members 16 in an appropriate position.

In either case (as shown most clearly in FIG. 8), a cavity 34 will result for each coupling member 16 to envelop the base 22 and, if desired, a portion of the shaft 18. This effectively maintains each coupling member 16 in an appropriate position for proper deployment. In a preferred embodiment, this “appropriate position” is one in which the coupling members 16 are disposed within the wall of the balloon 32 such that the penetrating tips 20 are pointing in a generally radially outward manner. Upon inflation and expansion of balloon 32, the coupling members 16 will be driven outwardly such that the penetrating tips 20 pierce through the bio-lining 14 and become engaged with the structural element 12. The inflation of the balloon 32 will simultaneously serve to loosen or dislodge the bases 22 from within the balloon cavities 34. The balloon 32 may thereafter be deflated and removed along with the rest of the catheter 30, leaving the bio-lining 14 securely coupled within the structural element 12.

Although shown in a specific configuration in FIGS. 3-8, it will be appreciated that coupling members 16 may be arranged in any number of different fashions and provided in varying quantities and/or dimensions depending upon the application. For example, although shown in FIG. 3 deployed in a plurality of rows, it will be appreciated that the coupling members 16 may be deployed in any number of different configurations, including but not limited to spiral, criss-cross, or randomly disposed. Coupling members 16 may also be provided in any number of different designs, such as those shown by way of example in FIG. 9.

Coupling members 16 may comprise any number of suitable biocompatible materials, including but not limited to polytetrafluoroethylene (PTFE), stainless steel, polyamides, polyimides, silicones, acrylates, methacrylates, fluorinated polymers, homopolymers, copolymers or polymer blends. Coupling members 16 may also comprise any of a variety of bio-degradable materials. An advantageous aspect of constructing coupling members 16 from bio-degradable material is that the (albeit modest) blood-device interface due to the bases 22 will be eliminated once the bio-degradation process is complete. Elimination of the bases 22 will also result in improved laminar blood flow, as described above with reference to the bio-degradable sutures 24 of FIG. 2.

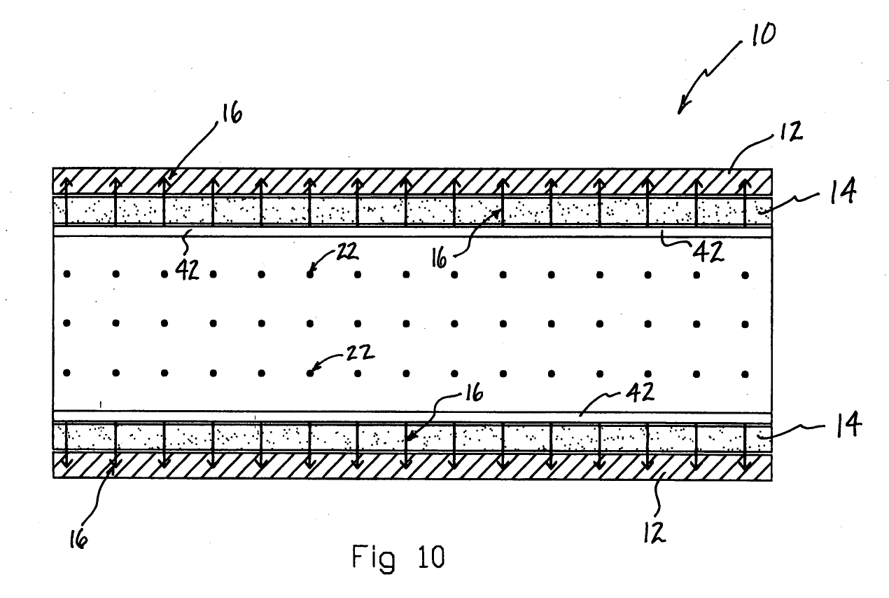

Although the coupling members 16 shown in FIGS. 3-9 are separate and distinct from each other (i.e. not interconnected), it is contemplated as part of the present invention to provide the coupling members 16 as part of a unitary structure such that the coupling members 16 are interconnected. For example, with reference to FIG. 10, the coupling members 16 can be formed as part of an inner structural element 42. Structural element 42 is preferably constructed according to the same principles set forth above with regard to structural element 12. That is, structural element 42 is preferably of a radially expandable construction such that it can be introduced within the bio-lining 14 while in a contracted state of minimal diameter and thereafter be deployed into an expanded state of increased diameter.

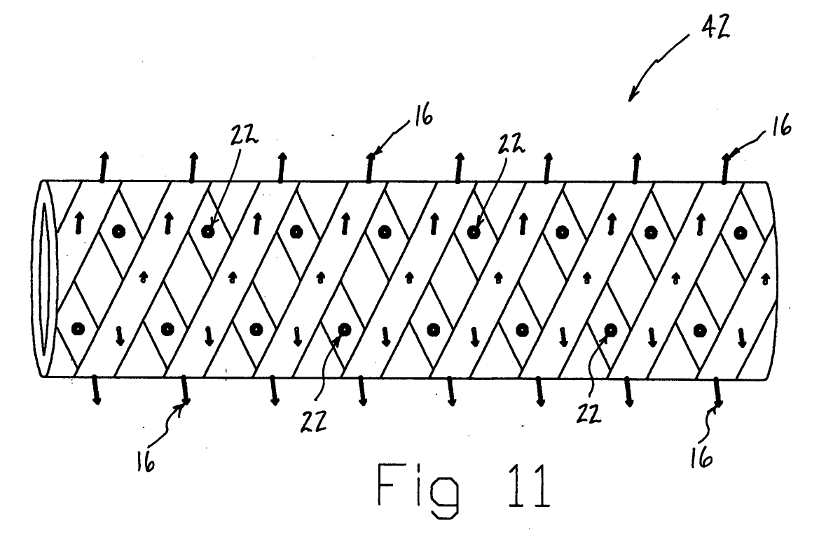

In one embodiment, the structural element 42 may comprise a stent having a generally tubular, meshed construction. Structural element 42 may comprise any number of suitable biocompatible materials, including but not limited to those enumerated above with reference to structural element 12. Although a blood-device interface does exist once the bio-lined structural element 10 is deployed within a treatment site, the meshed nature of such a stent-type structural element 42 minimizes the extent to which blood interfaces with the structural element 42. This, in turn, reduces the likelihood of restenosis within the treatment site. With reference to FIG. 11, this blood-device interface may be further reduced by providing the stent-type structural element 42 having a spiral construction.

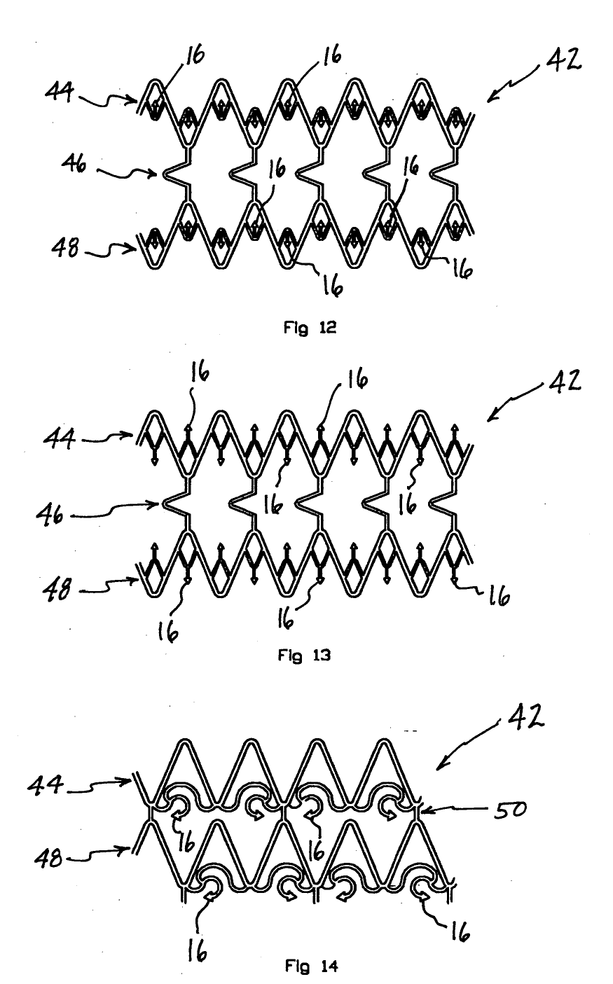

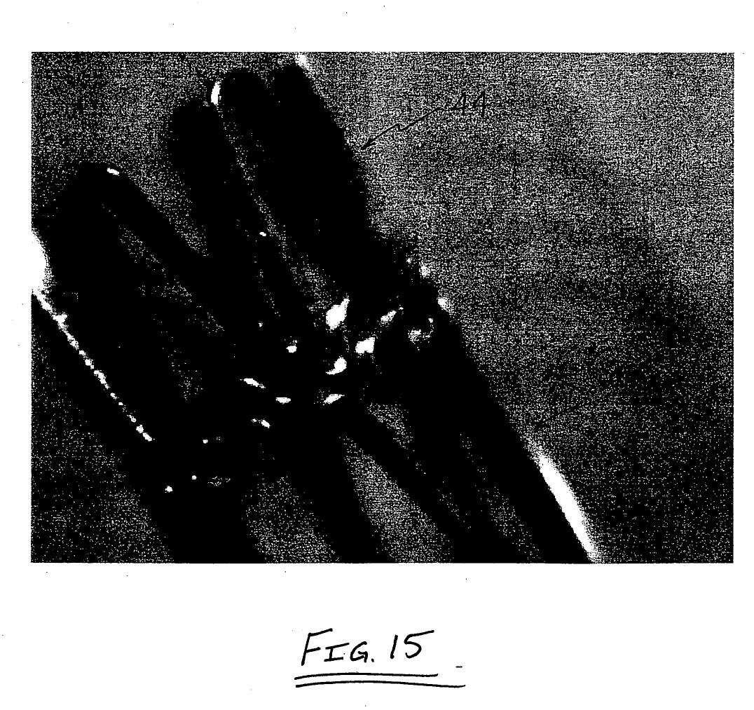

FIGS. 12-14 illustrate (by way of example only) various manners of constructing the stent-type structural element 42 according to a still further aspect of the present invention. For clarity, these stent designs are shown as if each stent-type structural element 42 were longitudinally cut and opened so as to lie flat within the plane of the paper. It will be appreciated, however, that each stent-type structural element 42 has a generally tubular shape in practice. Each stent-type structural element 42 is constructed having a plurality of interconnected “V” shaped elements 44, 46, 48 (and straight element 50 in FIG. 14). Whether balloon-expandable or self-expanding, each stent-type structural element 42 is constructed such that the coupling members 16 have a low-profile prior to deployment. That is, the coupling members 16 lie within the same general plane as the “V” shaped elements 44-50 prior to deployment.

Upon deployment, the “V” shaped elements 44-48 forming the stent-type structural element 42 will distend and become generally straightened. In an important aspect, this straightening of the “V” shaped elements 44-48 causes the coupling members 16 to extend generally perpendicularly from the generally cylindrical shape of the fully deployed stent-type structural element 42. In this fashion, each coupling member 16 will extend through the bio-lining 14 and engage with the mesh of the outer stent-type structural element 12 as shown in FIG. 10. The coupling members 16 may be provided in any number of different configurations, including but not limited to the “arrow-type” design shown in FIGS. 12-13 and the “hook-type” design shown in FIGS. 14-15.

B. Bio-Lining Preparation

As noted above, the bio-lining 14 may comprise any number of lining materials having characteristics that prevent or reduce the formation of vascular flow restrictions when deployed within a blood vessel. These materials include, but are not limited to, autologous vessel (harvested from the patient), tissue-engineered vessel (preferably based on the patient's own DNA), or synthetic vessel, or combination of any or all above-mentioned tissue. The following discussion sets forth, by way of example only, various manners of harvesting autologous tissue from the patient for use as the bio-lining 14 according to the present invention. It will be readily appreciated, therefore, that any number of different techniques for bio-lining preparation (i.e. using synthetic vessel and/or tissue-engineered vessel) may be employed without departing from the scope of the present invention. Moreover, it is to be readily understood that the following systems and methods of bio-lining preparation involving autologous tissue are set forth by way of example only.

1. Structural Element Implantation

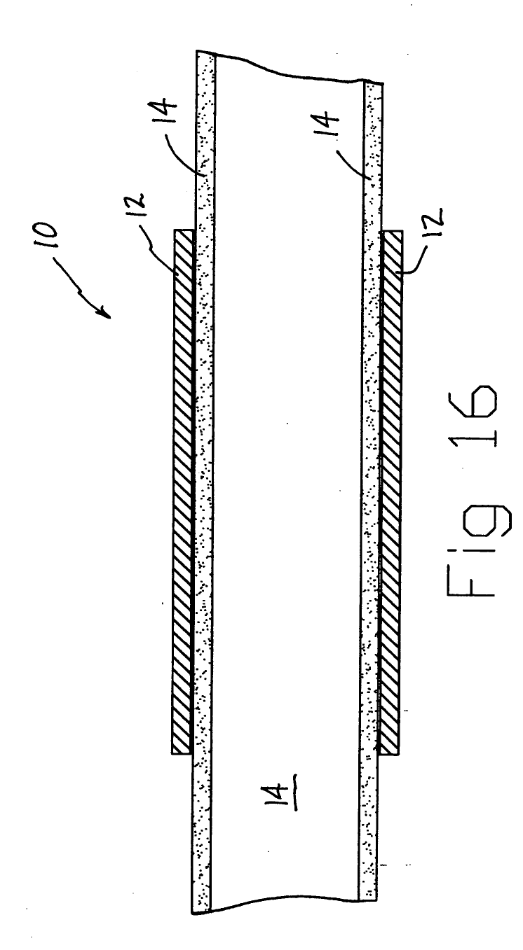

FIG. 16 illustrates one manner of preparing autologous bio-lining which involves implanting the structural element 12 over a blood vessel 14 within the patient for a sufficient duration such that the blood vessel 14 actually grows into (and becomes imbedded within) the structural element 12. This process may be undertaken in any number of different methods. One such method involves: (a) gaining access to a suitable blood vessel within the patient; (b) implanting the structural element 12 over the blood vessel; and (c) removing the structural element 12 from the patient after a sufficient duration has elapsed for the blood vessel 14 to have grown into (and become imbedded within) the structural element 12.

Step (a) of gaining access to a suitable blood vessel may be performed in any number of fashions, including but not limited to surgically cutting away various tissues or muscles in order to gain direct access to the given blood vessel. The blood vessel itself may include any number of suitable vessels within the patient, including but not limited to the radial artery and/or the internal mammary artery.

Step (b) of implanting the structural element 12 may be performed in any number of fashions, including but not limited to those involving severing the target blood vessel during implantation and those which leave the blood vessel undisturbed until the entire system (bio-lined structural element 10) is removed from the patient. The method involving severing the blood vessel may comprise the following steps: (i) severing the blood vessel at a single point along its exposed length; (ii) passing the structural element 12 over the severed blood vessel; and (iii) re-connecting (such as by suturing, surgical stapling, or other coupling devices) the ends of the severed blood vessel such that the structural element 12 is implanted over the blood vessel.

The method of implantation which leaves the blood vessel undisturbed (that is, non-severed) until eventual harvest may be accomplished in any number of different fashions. These include, but are not necessarily limited to, providing the structural element 12 such that it has a “placeable” design. As used herein, “placeable” is defined as any design that allows the structural element 12 to be positioned entirely or partially around the target blood vessel without first cutting or severing the target blood vessel. Such “placeable” structural elements 12 may include, but are not necessarily limited to, rollable stent devices of the type shown in U.S. Pat. No. 5,833,707 and stents or stent-type devices constructed from shape-memory materials such as Nitinol or shape-memory polymers described in U.S. Pat. No. 5,163,952 (the disclosures of both are hereby expressly incorporated by reference into this disclosure).

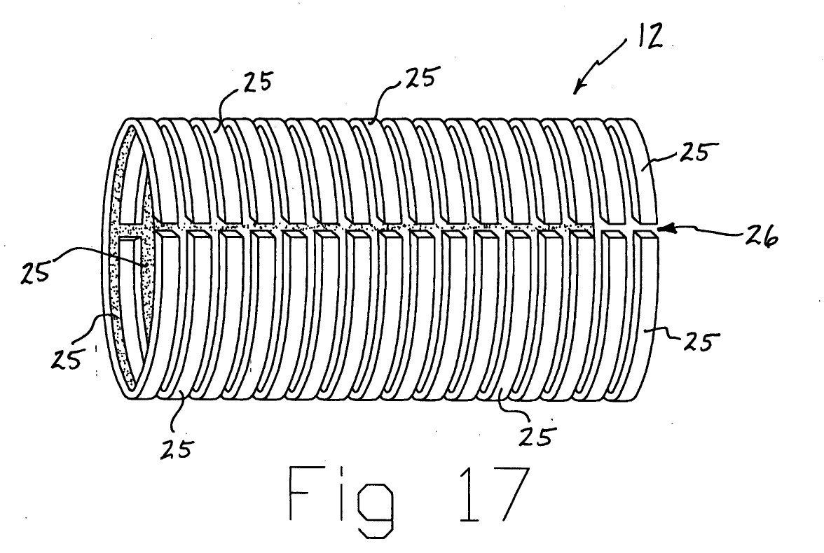

FIGS. 17-18 illustrate a still further manner of providing the structural element 12, featuring a semi-circular cross section comprised of (in FIG. 17) a plurality of arcuate members 25 and/or (in FIG. 18) a single coil-type arcuate member 25. According to one aspect of the present invention, the arcuate members 25 may be hinged (as in FIG. 17) or otherwise deformable (such as a coil-type arrangement in FIG. 18) such that they can be manipulated into position about the target vessel. The arcuate members 25 may also be dimensioned such that, when fully positioned about the target vessel, a channel or slot 26 is created between the ends of the arcuate members 25. Such a slot or channel 26 may be particularly advantageous in ensuring uninterrupted blood flow into side branches during the implantation period. That is, the structural element 12 may be positioned such that the side branches from the blood vessel extend through the slot 26. In so doing, the side branches will be free from impingement or crimping by the structural element 12, thereby ensuring uninterrupted blood flow.

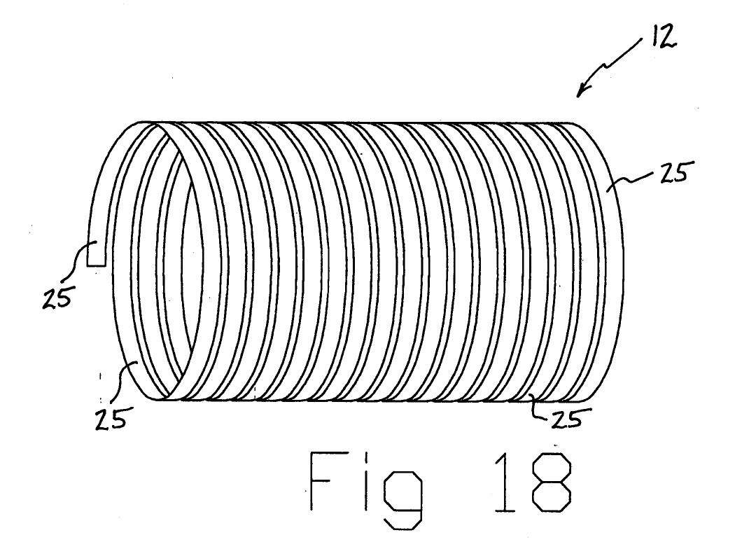

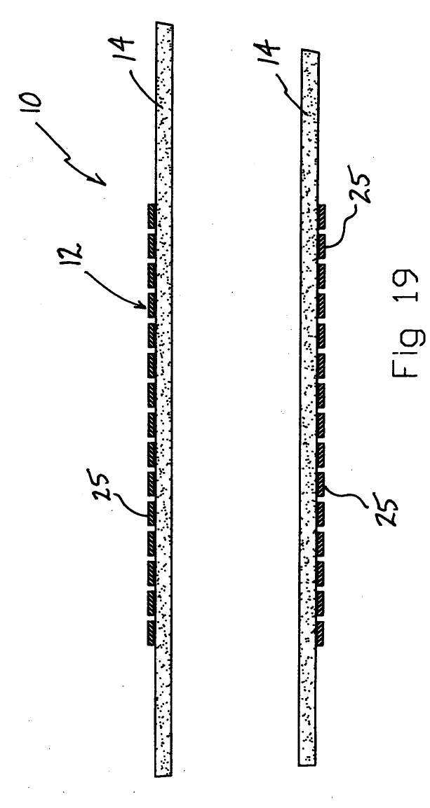

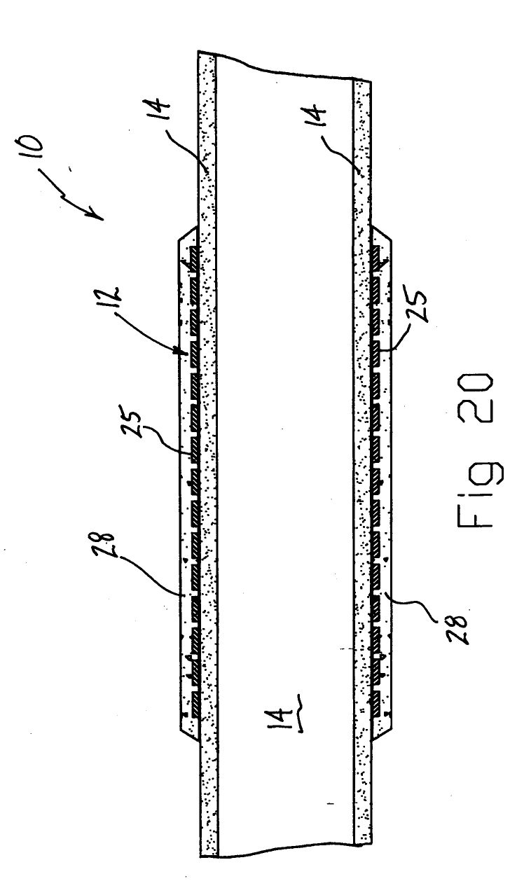

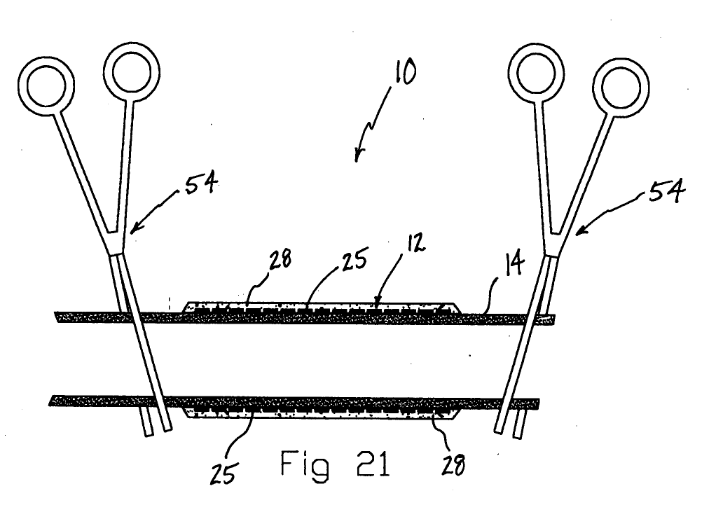

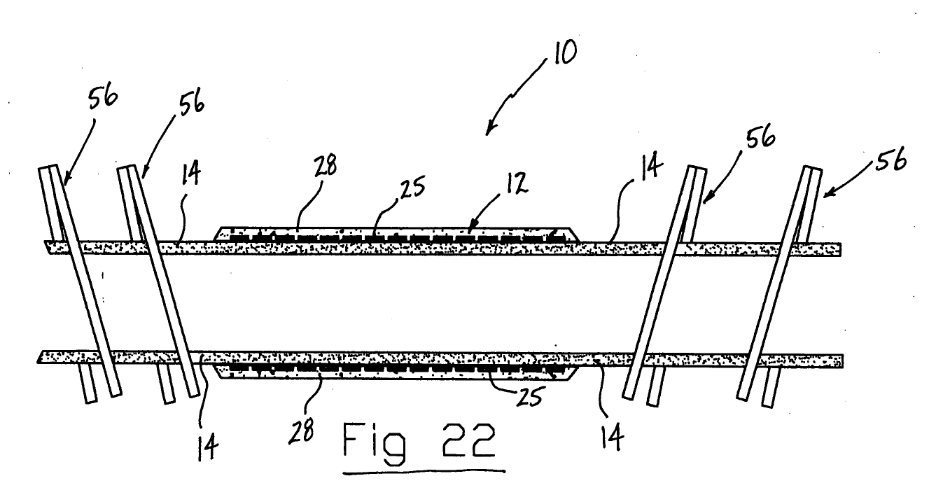

FIGS. 19-22 further illustrate the implantation and harvesting process according to the present invention. FIG. 19 shows the structural element 12 immediately upon implantation over the target vessel 14 (such as the radial artery). FIG. 20 shows the structural element 12 after the passage of time, wherein the target vessel 14 has grown into (and becomes imbedded within) the structural element 12. For clarity, the structural element 12 is shown with such tissue ingrowth 28 extending between the arcuate members 25. It will be appreciated, however, that such ingrowth will also take place into the actual arcuate members 25, particularly where the structural element 12 is provided as a stent or stent-type structure. FIGS. 21-22 shows two exemplary manners of removing the structural element 12 after tissue ingrowth has occurred. Namely, as shown in FIG. 21, scissors 54 may be employed to cut the blood vessel 14 on either side of the structural element 12. As shown in FIG. 22, this may also be accomplished through the use of surgical stapling devices capable of sealing off the blood vessel on either side of the structural element 12 with staples 56.

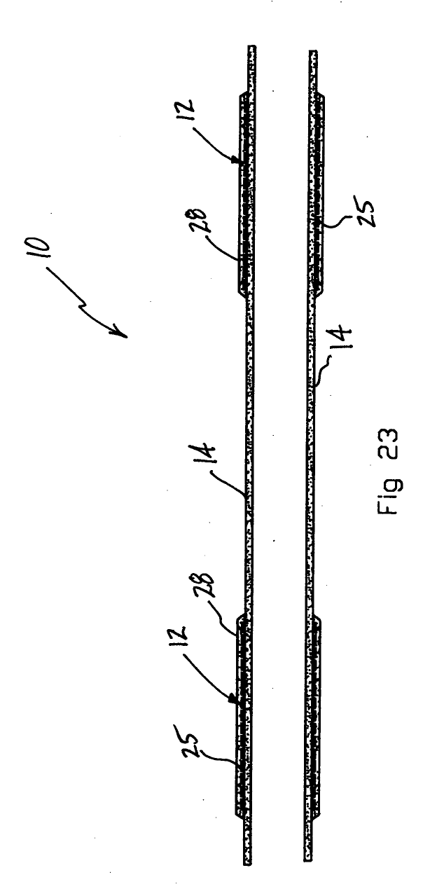

Although shown and described above with reference to a single structural element 12 for implantation, it is to be readily understood that the present invention clearly contemplates and covers the use of a plurality of structural elements 12 to overcome vascular flow restrictions. For example, as shown in FIG. 23, two separate structural elements 12 may be implanted over the target vessel 14 such that the structural elements 12 are separated by a predetermined distance. Following sufficient ingrowth, the structural elements 12 may be harvested from the patient such that a length of unsupported blood vessel 14 extends therebetween. One advantage of such a configuration is that, upon deployment into a region of vascular flow restriction, the unsupported region of the vessel 14 may be positioned within an existing structural device within the patient (such as a previously implanted stent).

With the autologous bio-lined structural element 10 harvested from the patient (regardless of the number of structural elements 12), the bio-lined structural element 10 may be implanted into a vessel experiencing restricted blood flow (such as a coronary artery). In this fashion, the blood flowing through the bio-lined structural element 10 will only contact the interior of the autologous bio-lining 14 within the structural element 12 and not the structural element 12 itself. This is a significant advantage over the prior art techniques for restoring blood flow in that it eliminates the interface between the blood and the diseased portion of the vessel or any foreign elements, thereby eliminating (or drastically reducing) the likelihood for restenosis.

2. Autologous Vessel Harvesting

The bio-lined structural element 10 of the present invention may also be prepared by harvesting a length of autologous blood vessel for immediate affixation within the structural element (as opposed to the longer duration implantation method described above). One such manner involves the use of a cutting catheter according to a still further aspect of the present invention. As will be described in greater detail below, the cutting catheter of the present invention may take any number of different forms. The common denominator between all these forms, however, is the inclusion of a cutting element that can be advanced over a length of autologous blood vessel and thereafter employed to harvest the autologous vessel for affixation within a structural element according to the present invention.

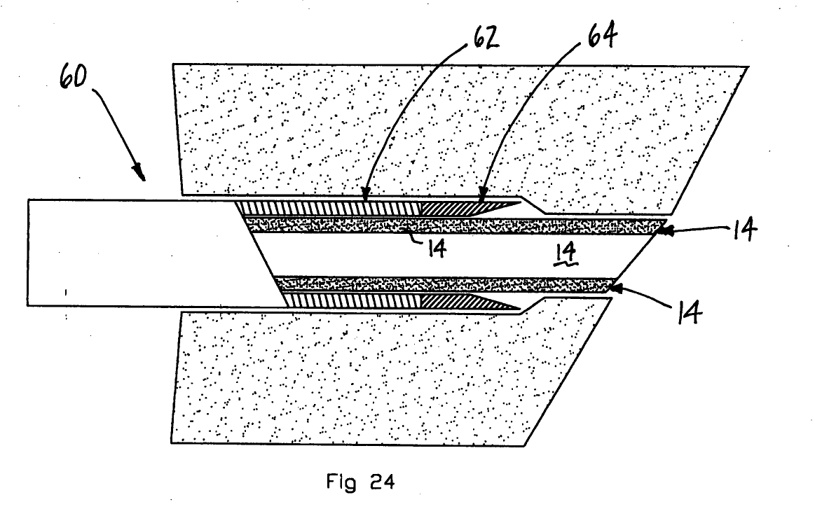

FIG. 24 illustrates, broadly, one such cutting catheter 60 according to the present invention. The cutting catheter 60 includes a catheter body 62 having a cutting element 64 disposed at or near the distal end. The catheter body 62 and cutting element 64 are dimensioned to be advanced over a length of autologous target vessel 14 such that the cutting element 64 progressively extricates the exterior surface of the blood vessel 14 from the surrounding tissues (not shown) within the patient. Following such extrication, the target vessel 14 may thereafter be harvested from the patient for use according to the present invention.

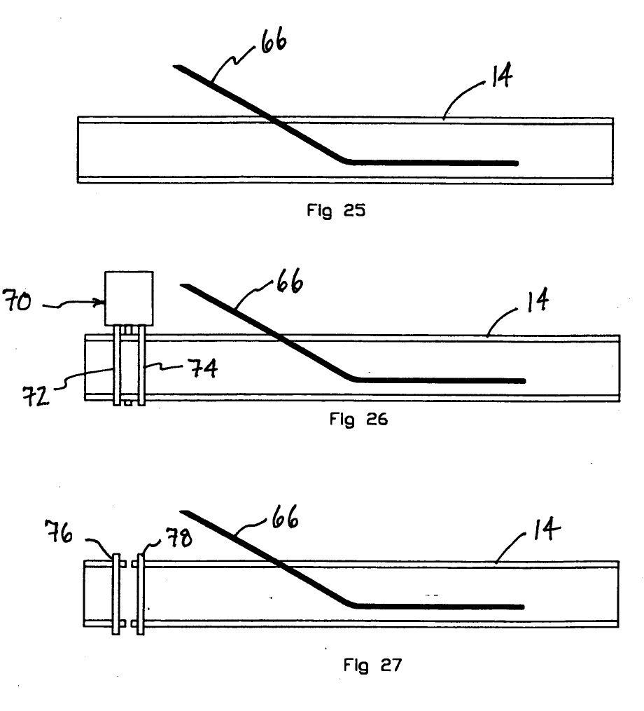

Various manners of positioning the cutting catheter 60 over the autologous tissue 14 will now be described. Referring to FIG. 25, a guide wire 66 may be employed to locate an area along the autologous blood vessel 14. The guide wire 66 may be introduced by advancing it through the layers of tissue surrounding the patient's target blood vessel 14 and onward through the wall of the blood vessel 14 for advancement into the interior lumen. The guide wire 66 is helpful in that it can be used to aid in the insertion of the cutting catheter 60 or other devices to the targeted vessel section 14.

With the guide wire 66 in place, a clip applicator 70 may then be employed to seal off the proximal end of the target vessel 14 as shown in FIGS. 26-27. The clip applicator 70 (shown by way of example only) may include a proximal clip applicator head 72 and a distal clip applicator head 74 for the purpose of applying proximal and distal clips 76, 78, respectively. A scissors (not shown) or similar cutting element on the applicator 70 may be employed to sever the target vessel 14 following the application of clips 76, 78 (FIG. 27).

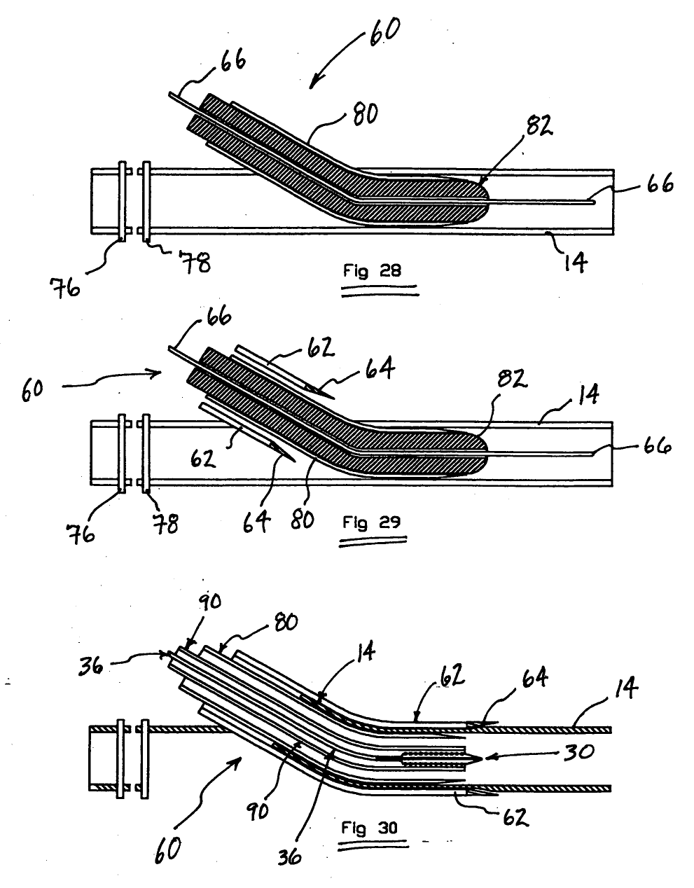

After locating the target vessel 25 and the placement of guide wires 20, an introducer 80 and a dilator 82 may then be advanced over the guide wire 66 into target vessel 14 as shown in FIG. 28. The introducer 80 comprises a generally tubular structure which, when positioned with its distal end within blood vessel 14, creates a port or lumen through which access may be gained into the interior of the blood vessel 14. The dilator 82 serves to expand the aperture formed by the guide wire 66 such that the introducer 80 may be passed into the interior of the blood vessel 14. Both the introducer 80 and dilator 82 may comprise any number of known or commercially available devices.

With reference to FIG. 29, the cutting catheter 60 may now be advanced over the introducer 80 to progressively extricate the exterior surface of the target vessel 14 from the surrounding tissue (not shown) according to the present invention. The cutting catheter 60 is advanced such that the cutting element 64 engages introducer 80 closely and follows its path. A small clearance exists between the cutting catheter 60 and introducer 80 which allows cutting catheter 60 to slide past introducer 80 while exerting minimal force on the cutting catheter 60. Cutting element 64 preferably contains a sharp blade portion 84 (such as the angled portion shown in FIG. 24) at or near its distal end. The angled nature of the blade portion 84 allows the cutting element 64 to closely follow the contour of the introducer 80 without cutting into or snagging on the exterior surface of the introducer 30.

As the cutting catheter 60 is advanced along the introducer 80, it will eventually force the cutting element 64 to cut through the wall of the blood vessel 14 as shown in FIG. 30. Once this occurs and the cutting catheter element 64 is positioned over the targeted vessel 14, dilator 82 may then be removed while keeping introducer 80 and guide wire 66 in place. A deployment catheter 30 may then be advanced over the guide wire 66 for the purpose of deploying a plurality of coupling members 16 maintained on the balloon 32 according to the same principles discussed above with reference to FIGS. 5-8. A protective sheath 90 may be employed to cover the coupling members 16 during advancement of the deployment catheter 30 within the target vessel 14. In this fashion, the coupling members 16 will not engage or impinge upon the interior of the blood vessel 14 until deployment.

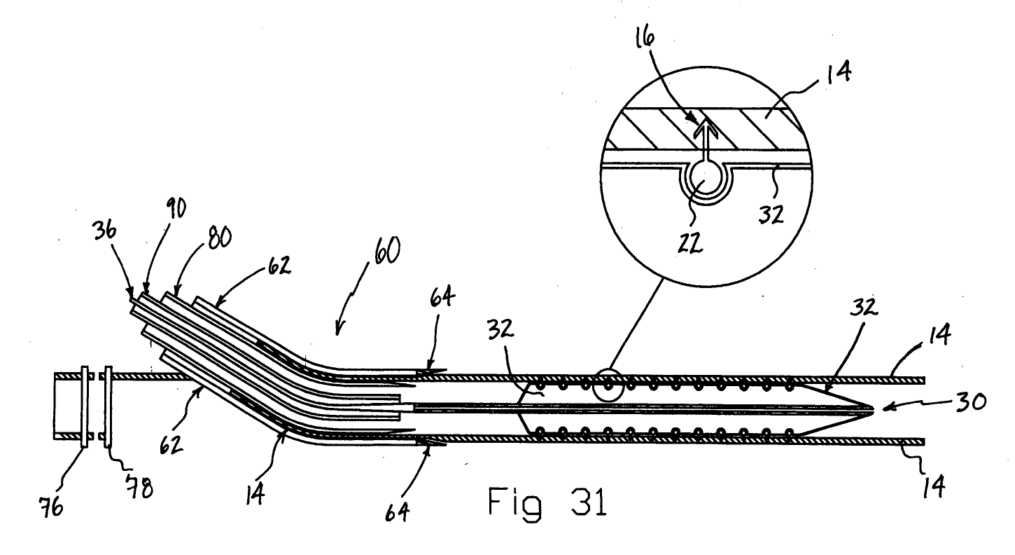

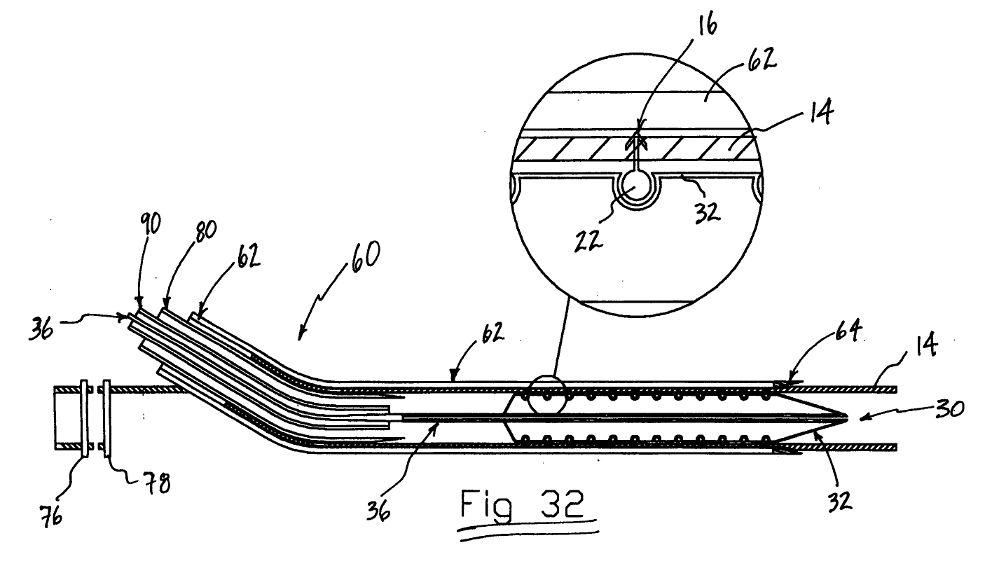

The deployment catheter 30 (preferably with protective sheath 90 in place) is thereafter advanced to a predetermined location within the blood vessel 14. With the deployment catheter 30 at this advanced location, the protective sheath 90 may be withdrawn and the balloon 32 inflated to thereby deploy the coupling members 16 as shown in FIG. 31. The cutting catheter 60 may then be advanced along the exterior surface of the blood vessel 14 to progressively cut the target vessel 14 from the surrounding tissue as shown in FIG. 32. In a beneficial aspect, this advancement is facilitated through the use of the penetrating tips 20, which preferably extend past the exterior periphery of the blood vessel 14 following deployment (best seen in FIG. 31). More specifically, the height of the penetrating tips 20 causes the cutting element 64 to “ride” over the penetrating tips 20, rather than on the exterior surface of the blood vessel 14 itself. This advantageously protects the exterior surface of the blood vessel 14 from being damaged, cut, or otherwise impinged by the cutting element 64. As will be appreciated, the angled nature of the blade portion 84 facilitates the progression along the penetrating tips 20.

With the coupling members 16 deployed into the blood vessel 14, and the blood vessel 14 extricated from the surrounding tissue, the distal end of the blood vessel 14 must then be cut or otherwise severed such that the blood vessel 14 may be withdrawn for use in lining a structural element 12 according to the present invention. Several illustrative cutting devices will be described below for accomplishing this task. At this point, however, it should be pointed out that any number of different manners, methods, or mechanisms may be employed to withdraw the blood vessel 14 from the patient, including but not limited to the deployment catheter 30 disclosed above, without departing from the scope of the present invention.

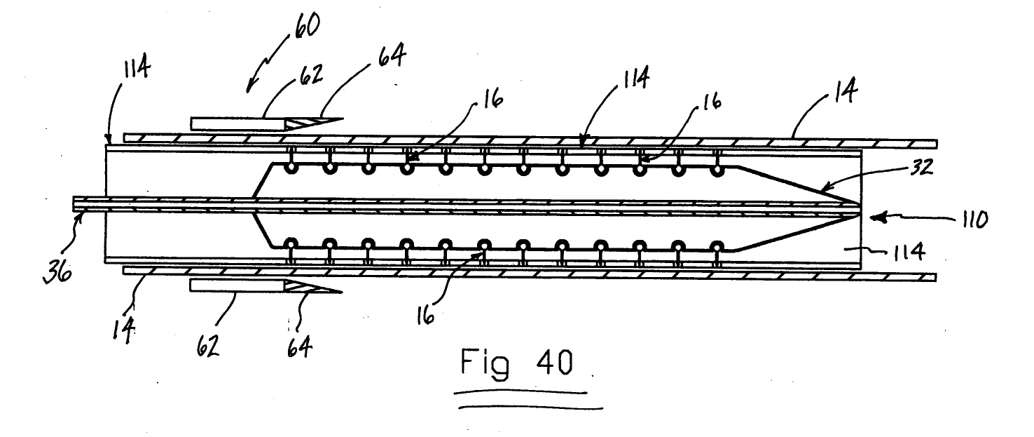

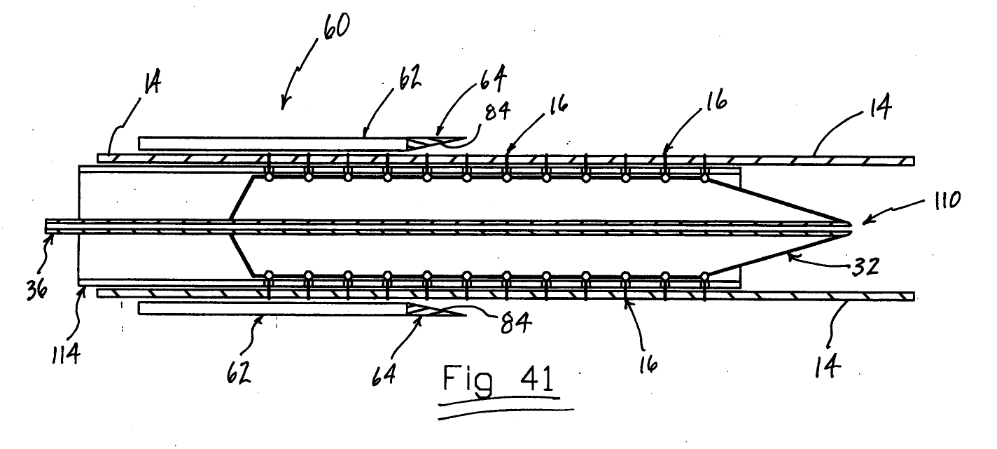

For example, with reference to FIGS. 40-41, a holding catheter 110 may be provided for temporarily holding the blood vessel 14 to accomplish such withdrawal. Holding catheter 110 may be constructed similarly to the deployment catheter 30, with a deployment balloon 32 disposed on the end of a catheter body 36 (preferably having a centrally disposed lumen for slideably receiving the guide wire 66). Unlike the deployment catheter 30, however, the coupling members 16 are fixedly attached to the balloon 32 such that they will not become physically removed or detached from the balloon 32 upon inflation. Moreover, the coupling members 16 are essentially straight and do not include any type of engagement tip (such as tip 20 disclosed above). An optional guide catheter 114 may be provided having apertures 112 suitable to pass and guide the coupling members 16 during expansion and contraction of the balloon 32.

Upon inflation, the coupling members 16 extend into the wall of the blood vessel 14 to thereby hold the blood vessel 14 in place (and at the same time protect the exterior surface of the blood vessel 14) while the cutting catheter 60 is employed as shown in FIG. 41. Once extricated from the surrounding tissue, the blood vessel 14 may be cut or severed in a manner to be described below, allowing the holding catheter 110 to be withdrawn from the patient with the blood vessel 14 temporarily maintained on the balloon 32. Once harvested, the blood vessel 14 may be removed or otherwise released by simply deflating the balloon 32 (causing the coupling members 16 to retract). The blood vessel 14 may thus be harvested from the patient in a quick and easy fashion for later affixation within a structural element 12 according to the present invention.

It should be readily appreciated that the features of the holding catheter 110 may be accomplished in any number of suitable fashions without departing from the scope of the present invention. For example, although shown disposed within the optional guide catheter 114, it will be appreciated that the feature of temporarily deploying the coupling members 16 may be accomplished without employing the guide catheter 114. That is, the guide catheter 114 need not be included if holding catheter 110 (via the expansion of balloon 32) is capable, by itself, of temporarily holding the blood vessel 14 according to the present invention.

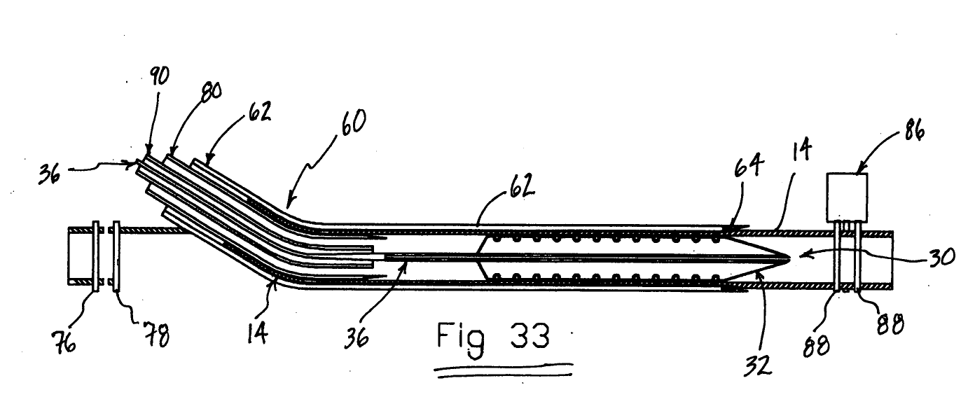

With the blood vessel 14 extricated from the surrounding tissue according to the present invention, the next step involves cutting the distal end of the targeted vessel 14 such that it can be physically removed from the patient for use as bio-lining within a structural element 12 according to the present invention. More specifically, cutting the end of the blood vessel 14 will allow the withdrawal of the entire harvesting assembly. This cutting step may be performed in any number of suitable fashions. One such method (shown generally in FIG. 33) involves inserting a cutting device 86 directly through the tissue above the distal end of the cutting catheter 60. By introducing the cutting device 86 in this fashion, and thereafter manipulating its cutting elements (shown generally at 88), the distal end of the blood vessel 14 may be severed for graft removal. Other methods may be employed which involve equipping the cutting catheter 60 with additional cutting features capable of severing the distal end of the blood vessel 14. For example, the cutting catheter 60 may be equipped with one or more apertures near its distal end capable of being employed to position an electrocautery snare (not shown) around the blood vessel 14. Once positioned around the blood vessel 14, the electrocautery snare may be selectively energized to sever or otherwise cut of the blood vessel 14 such that it can be removed from the patient.

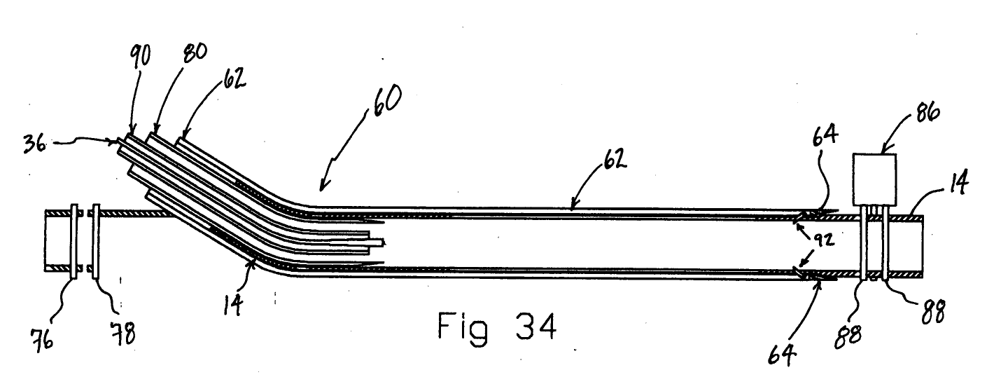

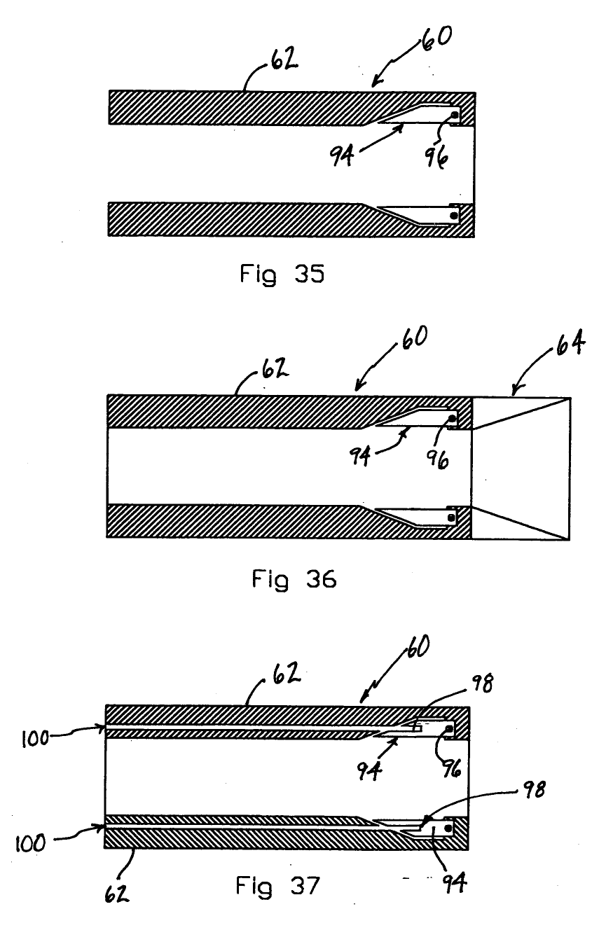

In a still further aspect of the present invention, the cutting catheter 60 may be equipped with one or more retractable cutting element(s) 94 as shown in FIGS. 34-37. Each retractable cutting element 94 is preferably hingedly disposed (via, for example, pivot pin 96) within a recessed portion along the interior of the catheter body 62. This positioning allows each retractable cutting element 94 to remain flush along the interior of the catheter body 62 as the cutting catheter 60 is advanced along the blood vessel 14. The hinged nature of each retractable cutting element 94 allows it to pivot between a retracted position during forward displacement (left to right in FIGS. 35-37) and a deployed position during backward displacement (right to left in FIGS. 35-37). In the deployed position, the retractable cutting element 94 extends inwardly toward the center of the cutting catheter 60 and serves to cut the blood vessel 14 as the cutting catheter 60 is moved backwards and/or rotated. Although not shown, the cutting catheter 60 may include one or more inwardly protruding, fixed cutting element(s) extending from the interior surface of the catheter body 62 near the cutting element 64.

The cutting catheter 60 as shown in FIG. 35 is set forth by way of example only, and it is to be readily understood that various modifications or alterations may be undertaken without departing from the scope of the present invention. For example, with reference to FIG. 36, the cutting catheter 60 may further include the cutting element 64 of the type shown and described above. Moreover, although not shown, it is contemplated as part of the present invention to provide the retractable cutting element 94 and the cutting element 64 on two separate cutting catheters.

FIG. 37 illustrates yet another aspect of the cutting catheter 60 of the present invention. Namely, each retractable cutting element 94 is equipped with a locking groove or lumen 98 capable of receiving a wire (not shown) for the purpose of locking the cutting element 94 within the recessed portion of the catheter body 62. In this case, the catheter body 62 will need a corresponding groove or lumen 100 in order to receive the aforementioned wire for engagement within the locking groove 98 of the retractable cutting element 94. In order to deploy each cutting element 94, the wire must first be withdrawn from the locking groove 98. Thereafter, the cutting catheter 60 may be pulled backwards to deploy the cutting element 94 for cutting the blood vessel 14. It is also contemplated as part of the present invention to provide the cutting element 94 with a bias to extend inwardly when the wire is withdrawn from the locking groove 98. This may be accomplished through the use of springs in conjunction with the pivot pins 96, as well as via material selection (i.e. using nitinol or other shape-memory materials to construct the cutting element 94).

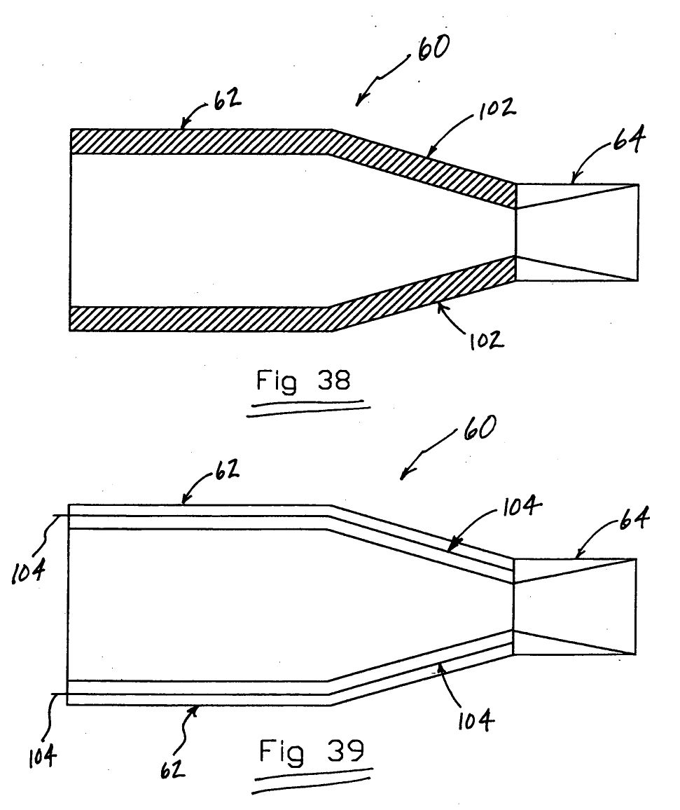

FIG. 38 illustrates a still further aspect of the cutting catheter 60 of the present invention. The cutting catheter 60 may be manufactured such that the main portion of the catheter body 62 and the cutting element 64 are of different diameter. One manner of accomplishing this is to provide the catheter body 62 with a tapered portion 102 extending between the main portion and the cutting element 64. A beneficial aspect of this design is that it minimizes (if not eliminates) the amount of drag experienced between the interior of the catheter body 62 and the exterior of the blood vessel 14. This reduction in drag improves the ease with which the cutting catheter 60 may be advanced over the blood vessel 14. This, once again, is based on the fact that the cutting element 64 is the only significant segment of the catheter 60 that contacts the blood vessel 14 during advancement. This is in contradistinction to cutting catheters of constant diameter (as shown above), which experience a dragging force along their entire length as they are advanced to cut the blood vessel 14 from surrounding tissue.

The cutting element 64 shown and described above with reference to FIGS. 24-38 may also take a number of different configurations without departing from the scope of the present invention. For example, the cutting element 64 may take any number of different shapes other than the angled configuration shown and described above (forming blade portion 84). The cutting element 64 may also comprise any number of different types of cutting instrumentation. For example, with reference to FIG. 39, the cutting element 64 may comprise a cauterization tip or a harmonic scalpel activated electrically through an electric wire 104 disposed within the catheter body 62. When provided as a cauterization element, the cutting element 64 may be selectively activated to cauterize as it is advanced along the exterior of the blood vessel 14, thereby preventing or minimizing any bleeding that may otherwise result from the extrication of the blood vessel 14 from surrounding tissue. When provided as a harmonic scalpel, the cutting element 64 may be selectively activated to harmonically cut the blood vessel 14 away from surrounding tissue during advancement of the cutting catheter 60.

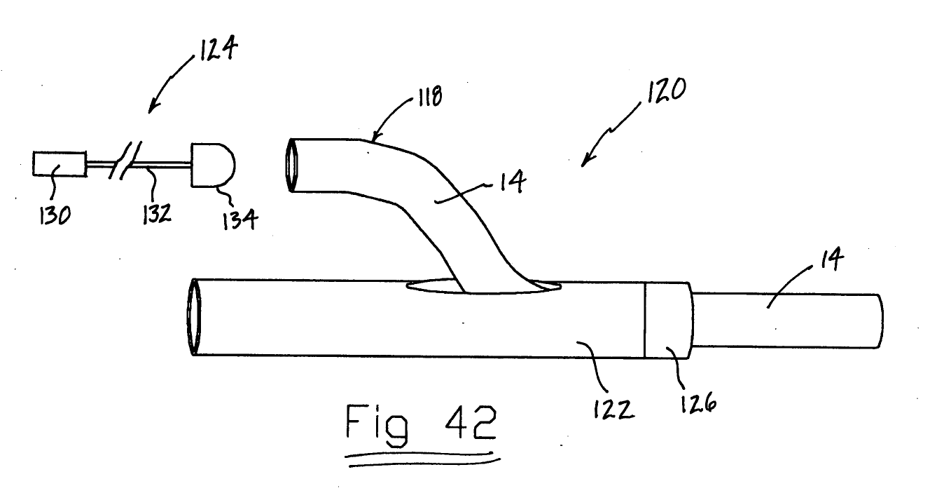

The foregoing manners and mechanisms for harvesting a length of blood vessel 14 are set forth by way of example only. For example, with reference to FIG. 42, a windowed cutting catheter 120 according to a still further aspect of the present invention is provided for extricating and cutting a length of blood vessel 14 from the surrounding tissue. The windowed cutting catheter 120 includes a catheter body 122 and an anvil assembly 124. The catheter body 122 is elongated, hollow and includes a cutting base 126 at its distal end and an access window 128 disposed a predetermined distance from the distal end. The lumen extending through the catheter body 122 and cutting base 126 is dimensioned such that a proximal portion 118 of the blood vessel 14 may be passed through the cutting base 126 and manipulated to exit out the access window 128 as shown. The catheter body 122 may be flexible but should preferably be of sufficient rigidity such that it can advance the cutting base 126 over the blood vessel 14. The cutting base 126 is preferably configured such that it extricates the blood vessel 14 from surrounding tissue during this advancement process. The windowed cutting catheter 120 is particularly suited for minimally invasive access. That is, a small incision may be made over a target blood vessel such that the target vessel can be cut, creating an open proximal end. The cutting base 126 may then be advanced over the proximal end of the blood vessel 14 until it exits the access window 128. At that point, the cutting base 126 may be advanced to “burrow” through the tissue surrounding the exterior of the blood vessel 14 to extricate the blood vessel 14 from surrounding tissue.

With the length of blood vessel 14 thus extricated, the anvil assembly 124 may then be employed to cut the distal end of the blood vessel 14 such that the blood vessel 14 may be removed for use in preparing a bio-lined structural element 10 according to the present invention. The anvil assembly 124 includes a handle member 130, a shaft 132 extending from the handle member 130, and an anvil member 134 disposed on the distal end of the shaft 132. In use, the anvil member 134 is introduced into the open proximal end 118 of the blood vessel 14 and advanced through the interior of the blood vessel 14 until it comes into contact with the cutting base 126. The cutting base 126 and anvil member 134 are dimensioned such that, when such contact is caused, the exterior of the anvil member 134 and the interior of the cutting base 126 cooperatively act to sever or cut the distal end of the blood vessel 14. With the distal end of the blood vessel 14 cut or severed, the anvil assembly 124 may be withdrawn from the catheter body 122 (such as by pulling it through the access window 128). The blood vessel 14 may then be removed from its position over the shaft 132 and employed to form the bio-lined structural element 10 according to the present invention.

3. Tissue Engineering

The bio-lined structural element 10 of the present invention may also be produced by equipping a structural element 12 with a bio-lining created through tissue-engineering techniques. Such tissue-engineering techniques are described, among other places, by L'Heureux et al. in “A Human Tissue-Engineered Vascular Media: A New Model for Pharmacological Studies of Contractile Responses” (FASEB J. 2001 February; 15(2): 515-24), Michel et al. in “Characterization of a New Tissue-Engineered Human Skin Equivalent with Hair” (In Vitro Cell Dev. Biol. Anim. 1999 June; 35(6): 318-26), and L'Hereux et al. in “In Vitro Construction of a Human Blood Vessel from Cultured Vascular Cells: A Morphologic Study” (J Vasc Surg 1993 March; 17(3): 499-509, the contents of which are hereby incorporated by reference as if set forth fully herein.

These tissue-engineering techniques may be used according to the following method of the present invention: (a) obtaining a tissue sample from a patient; (b) growing a length of tissue-engineered bio-lining based on the sample; and (c) equipping a structural element 12 with the tissue-engineered bio-lining to produce the bio-lined structural element 10. Step (c) may be performed by affixing or otherwise securing the tissue-engineered bio-lining within the structural element 12 in any number of suitable fashions, including but not limited to those described herein.

One advantage of this method is that the patient may undergo the tissue sample retrieval during an initial visit and thereafter have the complete bio-lined structural element 10 implanted during a later, subsequent visit. That is to say, the tasks of growing the tissue-engineered bio-lining 14 and securing it within the structural element 12 may be performed “off-line” such that the patient need only be present for tissue-sample retrieval and implantation of the completed bio-lined structural element 10. This advantageously minimizes the amount of time the patient will need to be hospitalized or present in a clinic for treatment of a vascular flow restriction.

II. Vessel Buttress

A bio-lined structural element according to the present invention may also be produced by disposing a structural element about some or all of the periphery of a vessel suffering from a vascular flow restriction and thereafter affixing the structural element to the native vessel. By buttressing the vessel in this fashion, the lumen of the vessel suffering the vascular flow restriction may become “opened” or otherwise widened to increase the inner diameter, thereby producing improved blood flow. This concept of overcoming vascular flow restrictions according to the present invention may be accomplished in any of a variety of suitable fashions, including but not limited to the following exemplary configurations described below.

A. Semi-Arcuate Structural Element

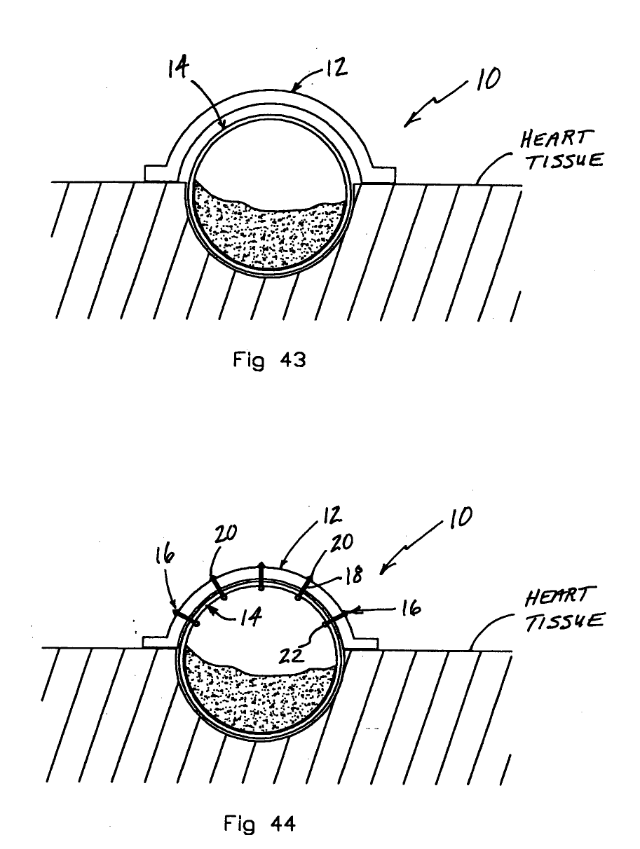

FIGS. 43-44 illustrate one such exemplary system for overcoming vascular flow restrictions according to the present invention. Namely, a semi-arcuate structural element 12 is provided over the exposed portion of the periphery of a blood vessel 14 which, in this case (by way of example only) is a coronary artery. Once the semi-arcuate structural element 12 is disposed in this position, a plurality of coupling members 16 may be employed to pierce through the coronary artery 14 such that the base 22 of each coupling member 16 is within the lumen of the coronary artery 14 and each penetrating tip 20 is disposed on the exterior surface of the arcuate member 12. As one skilled in the art will appreciate, the coupling members 16 may be deployed in this fashion using a deployment balloon 32 similar to, if not identical, to that shown and described above with reference to FIGS. 5-8. That is, the deployment balloon 32 may be introduced into the target site via percutaneous techniques. Once positioned under the structural element 12, the coupling members 16 may be deployed to thereby expand the inner diameter of the blood vessel 14 and, moreover, to maintain it in this expanded state (by affixing it to the interior of the structural element 12) for improved blood flow.

The structural element 12 may take the form of any number of suitable materials and shapes. For example, the structural element 12 may be essentially straight or curved and have a length suitable to cover some or all of the length of the vascular flow restriction. Those skilled in the art will also appreciate that the manner of expanding and affixing the coronary vessel 14 to the structural element 12 may be accomplished in any number of suitable fashions, rather than through the use of coupling members 16, without departing from the scope of the present invention. For example, any number of adhesives could be employed along the exterior surface of the vessel wall such that, when brought into contact with the inner surface of the structural element 12, the vessel wall may be caused to remain in this expanded position. That, for example, may occur through the use of an expansion balloon 32 within the lumen of the vessel 14 to maintain the vessel wall in contact with the interior surface of the structural element 12 for a sufficient duration to effect curing of the adhesive (such as through the use of UV-activated adhesive).

B. Generally Cylindrical, Hinged Structural Element

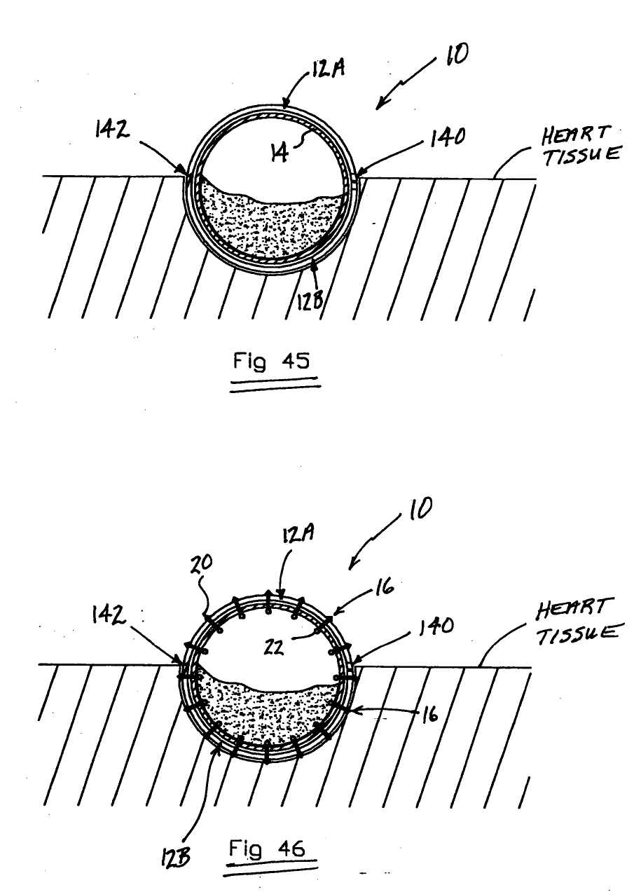

FIGS. 45-46 illustrate a still further exemplary system for overcoming vascular flow restrictions according to the present invention. In this case, a generally cylindrical structural element 12 of hinged construction is disposed over a vascular treatment site of, for example, a coronary artery 14. More specifically, the generally cylindrical structural element 12 comprises a pair of arcuate members 12A, 12B which are hingedly coupled via at least one hinge element 140 and optionally locked or otherwise closed together via a clasp member 142. The hinged coupling allows the arcuate members 12A, 12B to be temporarily separated or “opened” such that the free end of one of the arcuate members (i.e. 12B in FIG. 45) may be passed or burrowed under the lower or non-exposed periphery of the coronary artery. Once arcuate member 12B is passed under the coronary artery 14, the arcuate member 12A may be “closed” or brought into contact with arcuate member 12B, thereby encompassing the target area of the coronary artery 14 within the generally cylindrical structural element 12 (as shown in FIG. 45). As will be appreciated, the clasp member 142 may be omitted, particularly if the structural elements 12A, 12B are biased into a normally closed position.

With the generally cylindrical structural element 12 disposed in this position, a plurality of coupling members 16 may be employed to pierce through the coronary artery 14 such that the base 22 of each coupling member 16 is within the lumen of the coronary artery 14 and each penetrating tip 20 is disposed on the exterior surface of the arcuate members 12A, 12B (as shown in FIG. 46). As one skilled in the art will appreciate, the coupling members 16 may be deployed in this fashion using a deployment balloon 32 similar to, if not identical, to that shown and described above with reference to FIGS. 5-8. That is, the deployment balloon 32 may be introduced into the target site via percutaneous techniques. Once positioned under the structural element 12, the coupling members 16 may be deployed to thereby expand the inner diameter of the blood vessel 14 and, moreover, to maintain it in this expanded state (by affixing it to the interior of the structural element 12) for improved blood flow.

As with the structural element 12 in FIGS. 43-44, the generally cylindrical structural element 12 may take the form of any number of suitable materials and shapes. For example, the structural element 12 may be essentially straight or curved and have a length suitable to cover some or all of the length of the vascular flow restriction.

Although shown going from “inside-out” in FIG. 46, it will be appreciated that the coupling members 16 may be deployed from “outside-in” such that the base members 22 rest against the exterior surface of the structural element 12 and the penetrating tips 20 are disposed on the inside of the blood vessel 14. This manner of deployment may be facilitated by positioning an “anvil member” or similarly solid structure within the blood vessel 14 such that the penetrating tips 20 will become expanded or bent upon contact therewith, thus aiding to secure the blood vessel 14 to the structural element 12.

Any number of adhesives may be employed along the exterior surface of the vessel wall 14 such that, when brought into contact with the inner surface of the structural element 12, the vessel wall 14 may be caused to remain in this expanded position. These adhesives may include, but are not necessarily limited to, UV-activated adhesives.

A still further manner of coupling or otherwise affixing the blood vessel to the generally cylindrical structural element 12 involves the use of coupling members 16 formed as part of a unitary structure, such as an inner structural element 42 of the type shown and described with reference to FIG. 10-15. In such an arrangement, the inner structural element 42 may be selectively positioned within the target area within the vessel and thereafter deployed (such as by balloon expansion) such that the coupling members 16 pierce through the wall of the blood vessel 14 and into the generally cylindrical structural element 12. In this fashion, the blood vessel 14 will be secured within the interior surface of the structural element 12 in an expanded state for improved blood flow.

III. Bio-Lining with Structural Element Terminations

Vascular flow restrictions may also be overcome according to the present invention by providing a pair of bio-lined structural elements disposed a distance from one another and connected by a length of bio-lining. In this fashion, each of the bio-lined structural elements may be deployed on either side of a vascular flow restriction such that flow is restored through the length of bio-lining that extends therebetween. This concept of overcoming vascular flow restrictions according to the present invention may be accomplished in any of a variety of suitable fashions, including but not limited to the following exemplary configurations described below.

A. Two Piece Bio-Lining

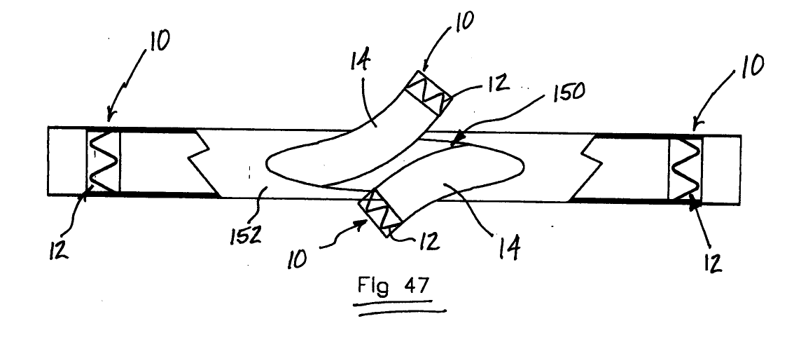

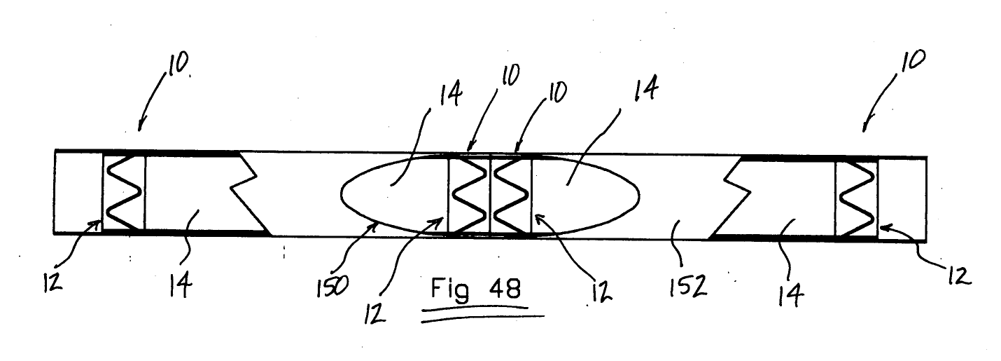

FIGS. 47-48 illustrate one such exemplary system for overcoming vascular flow restrictions according to the present invention. Namely, a pair of bio-lined structural elements 10 are provided, each having a length of bio-lining 14 extending therefrom. The structural elements 10 are preferably dimensioned to be introduced into an incision 150 formed through the wall of a vessel 152 suffering from a vascular flow restriction. Thereafter, each structural element 12 may be deployed in order to secure each bio-lined structural element 10 on either side of the incision 150. The structural elements 12 may comprise any number of suitable structures, including those described above and, most preferably, of balloon-deployable construction such that they may be introduced through the incision via a balloon catheter, which may thereafter be used to deploy the structural element 12. Once deployed in the target vessel 152, the free end of each length of bio-lining 14 may be connected together (as will be explained below) in order to provide a continuous lumen between the bio-lined structural elements 10 as shown in FIG. 48. As will be appreciated, this system advantageously provides direct access to remove the vascular flow restriction (through the incision 152) and thereafter provides the ability to quickly and easily restore a path of fluid communication for improved blood flow.

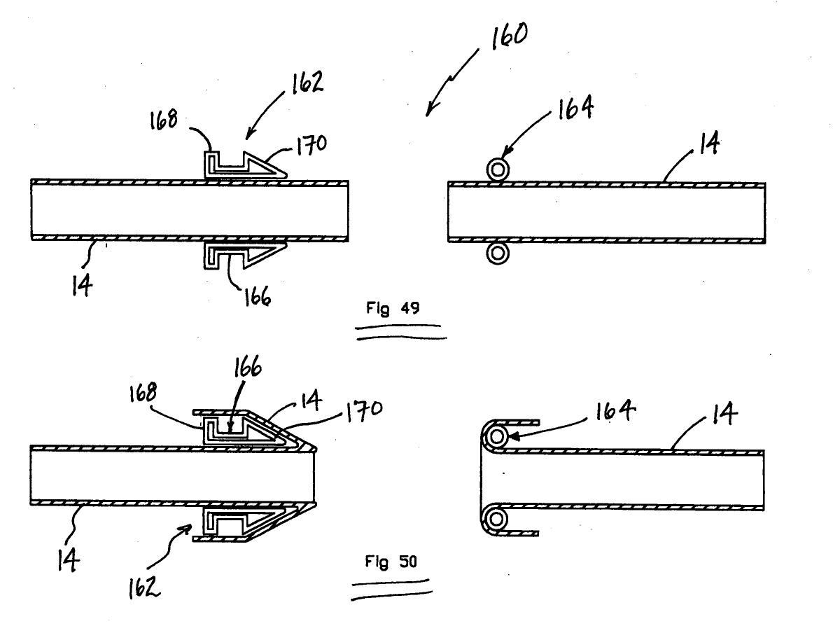

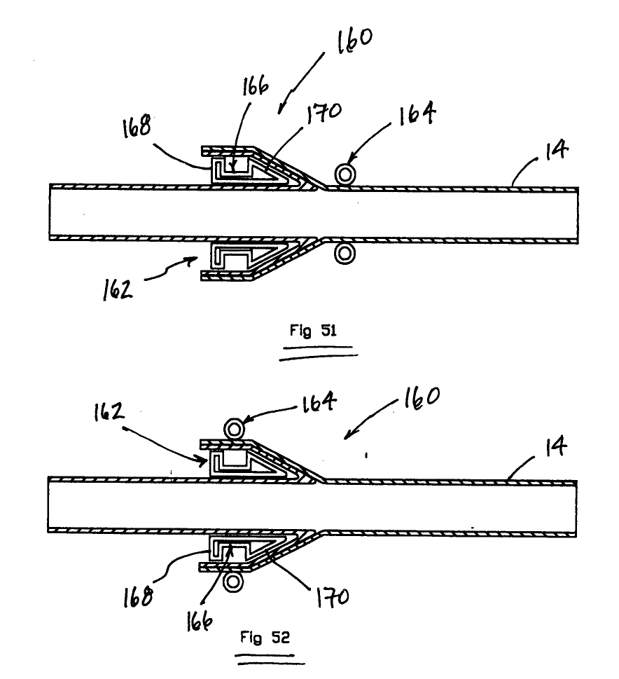

The free ends of each length of bio-lining 14 may be coupled together or otherwise connected in any of a variety of suitable fashions without departing from the scope of the present invention. For example, with reference to FIGS. 49-52, a lap joint assembly 160 may be employed according to one aspect of the present invention. Lap joint assembly 160 includes a connector member 162 and a ring member 164. The connector member 162 has a groove 166 formed between a ridged portion 168 and an angled portion 170. The ring member 164 is generally elastic and dimensioned to engage within the groove 166 of the connector 162 to secure the free ends of the bio-lining 14. As shown in FIG. 49, both the connector 162 and ring member 164 have an inner lumen dimensioned to pass a respective free end of bio-lining 14 therethrough. As shown in FIG. 50, each free end is thereafter rolled back over the respective portion of the lap joint assembly 160. From this point, as shown in FIG. 51, the ring member 164 and connector 162 are brought into close proximity and the free end from the ring member 164 is rolled off and extended over the free end residing over the connector 162. The ring member 164 may thereafter be rolled over the angled portion 170 of the connector 162 and, in so doing, come to rest within the groove 166. coupling the bio-linings 14 in this fashion thus advantageously restores blood flow and minimizes, if not eliminates, any blood-device interface.

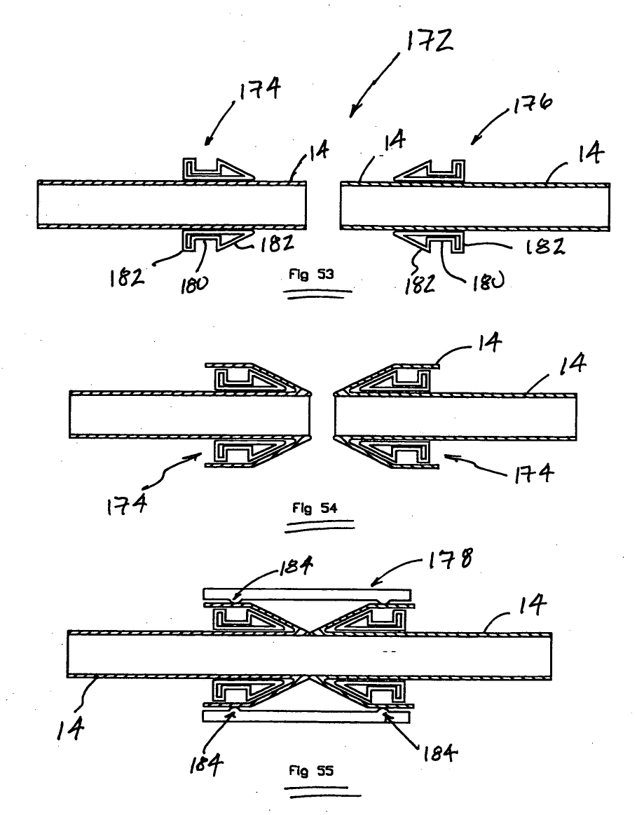

A still further exemplary manner of coupling or otherwise connecting the free ends of the bio-lining 14 is shown with reference to FIGS. 53-55. A butt joint assembly 172 is provided having a pair of connector bases 174, 176 and a connector shell 178. Each connector base 174, 176 includes a groove 180 formed between a pair of ridged portions 182. The connector shell 178 includes a pair of ridged portions 184 which are capable of being lodged within the groove portion 180 of a respective connector base 174, 176. As shown in FIG. 53, each connector base 174, 176 includes an inner lumen dimensioned to pass a respective free end of the bio-lining 14 therethrough. Each free end is thereafter rolled over the respective connector base 174, 176, preferably such that the free end is disposed at least partially within the respective groove 180 as shown in FIG. 54. At that point, each connector base 174, 176 may be urged or otherwise advanced into the connector shell 178 such that the ridged portions 184 of the connector shell 178 engage within the groove 180 of the respective connector base 174, 176 as shown in FIGS. 54 and 55. Once again, the resulting lumen of bio-lining 14 thus advantageously restores blood flow and minimizes, if not eliminates, any blood-device interface.

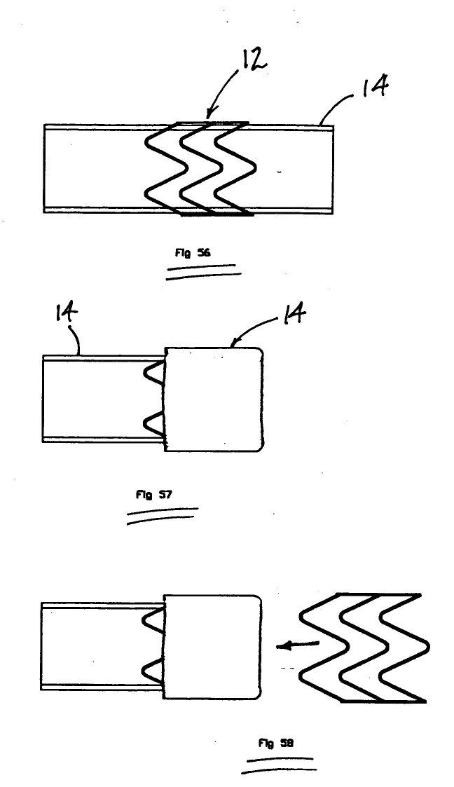

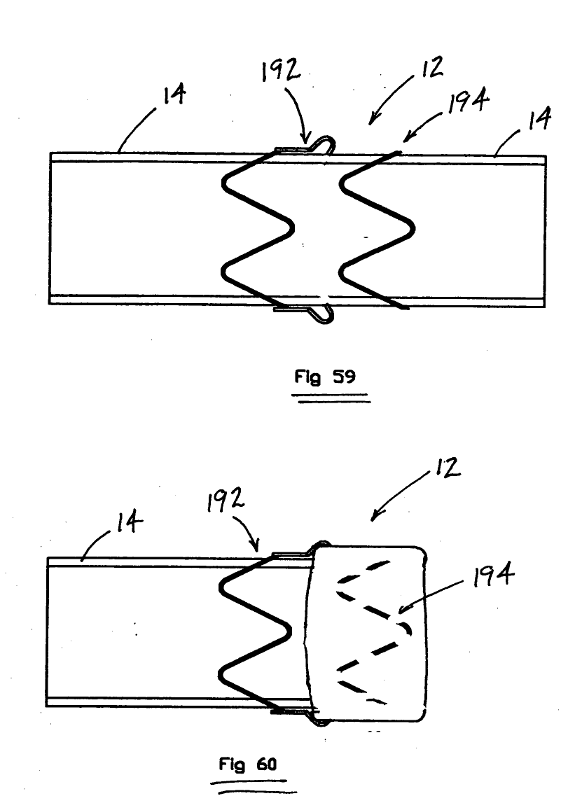

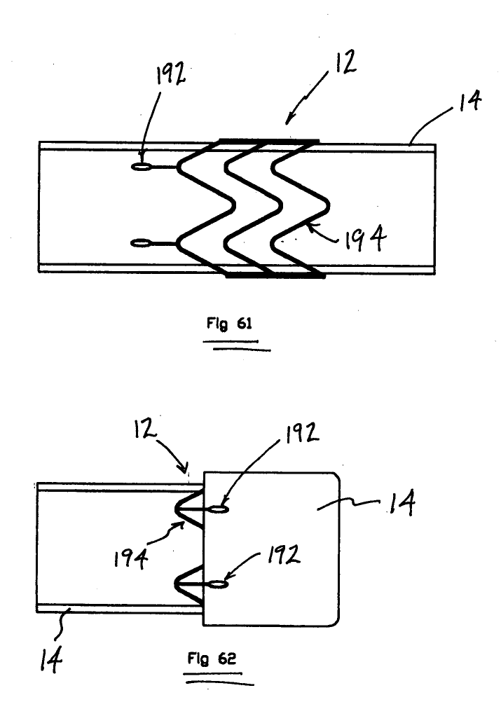

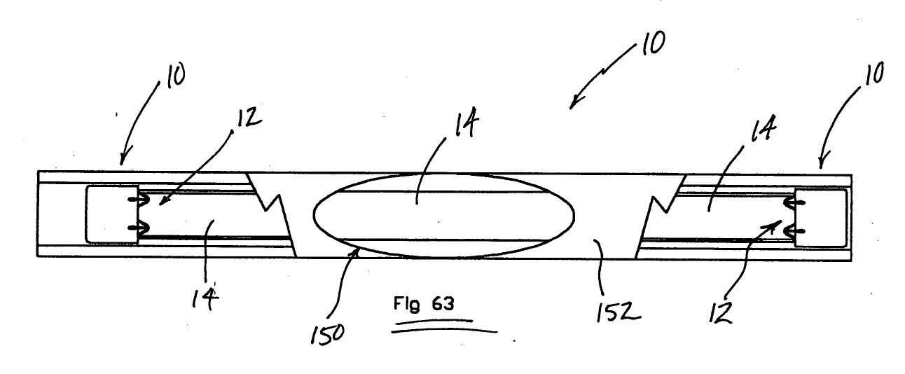

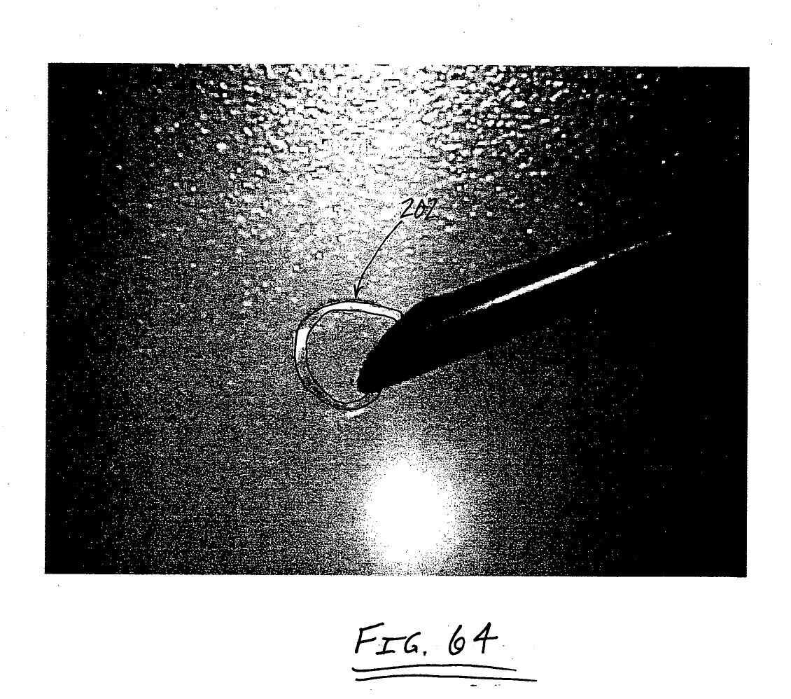

As mentioned above, each structural element 12 forming part of the embodiment shown in FIGS. 47-55 is preferably of balloon-expandable construction. FIGS. 56-62 illustrate exemplary manners of securing bio-lining 14 to each balloon-expandable structural element 12 according to the present invention. In one aspect, the free end of each length of bio-lining 14 (only one shown for clarity) may be passed through the lumen of structural element 12 (FIG. 56) and thereafter rolled over the structural element 12 (FIG. 57). For added purchase, a second (outer) structural element 190 may be placed over the first structural element 12 so as to sandwich the free end of the bio-lining 14 therebetween as shown in FIG. 58. Alternatively, each structural element 12 may be constructed having additional features for securing the free ends of the bio-lining 14. For example, with reference to FIGS. 59-60, each structural element 12 may be equipped with a plurality of members or extensions 192 capable of being bent inwardly towards the main body 194 of the structural element 12 to thereby close upon the bio-lining 14. In similar fashion, as shown in FIGS. 61-62, the structural element 12 may be provided with a plurality of members or extensions 192 capable of being folded over towards the main body 194 of the structural element 12 to thereby close upon the bio-lining 14.

B. One Piece Bio-Lining

FIG. 63 illustrates a still further exemplary system for overcoming vascular flow restrictions according to the present invention. In this embodiment, a pair of bio-lined structural elements 10 are provided, this time having a single length of bio-lining 14 extending between them. In order to provide a single lumen (as opposed to two separate lengths of bio-lining 14 as shown in FIGS. 47-55), at least one of the structural elements 12 must be of self-expanding construction. By way of example only, the proximal structural element 12 (on left in FIG. 63) may be of balloon-expandable construction and the distal structural element 12 (on right in FIG. 63) may be of self-expanding construction. Under this scenario, the proximal structural element 12 would be secured to the proximal free end of the bio-lining 14 in one of the manners described above with reference to FIGS. 56-62 and introduced into the incision 150 over a balloon catheter (not shown) for deployment upstream from the vascular restriction.

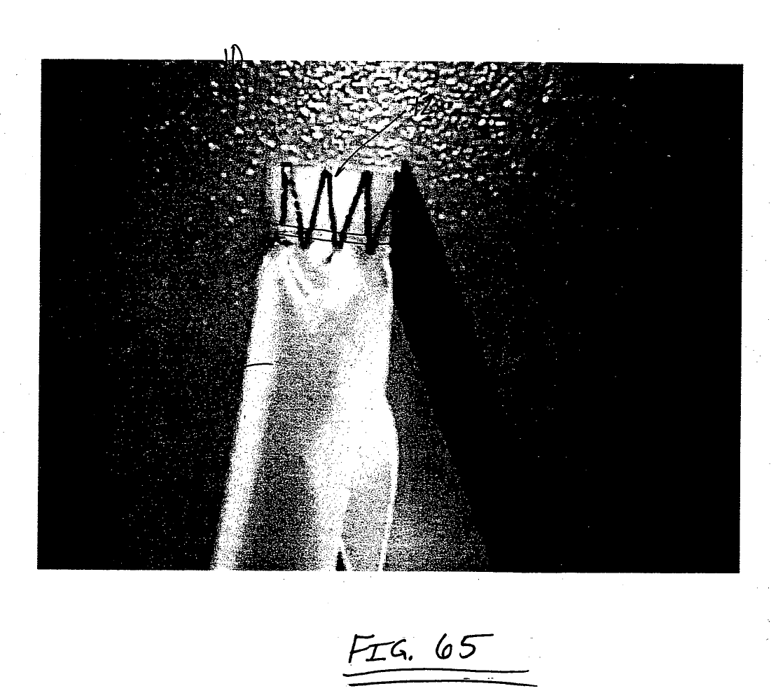

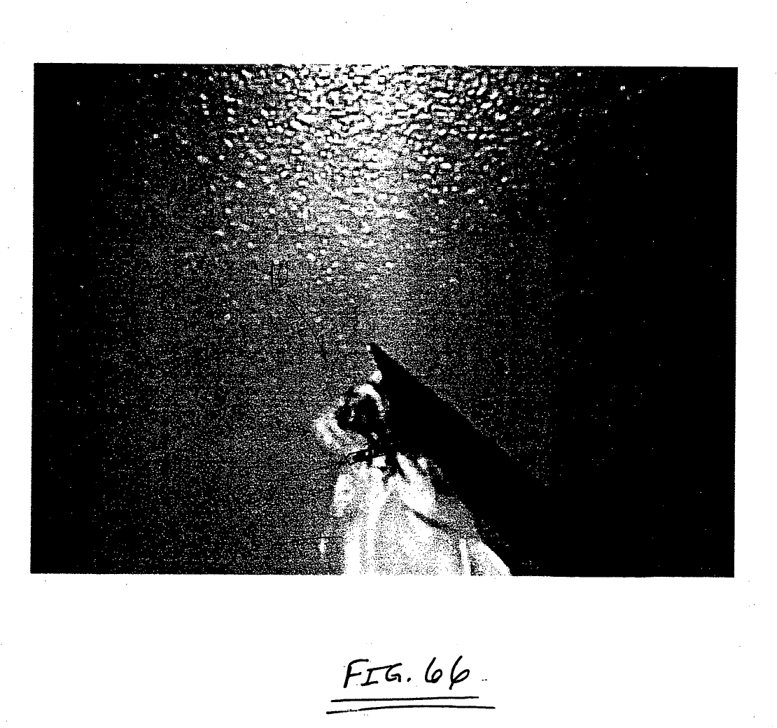

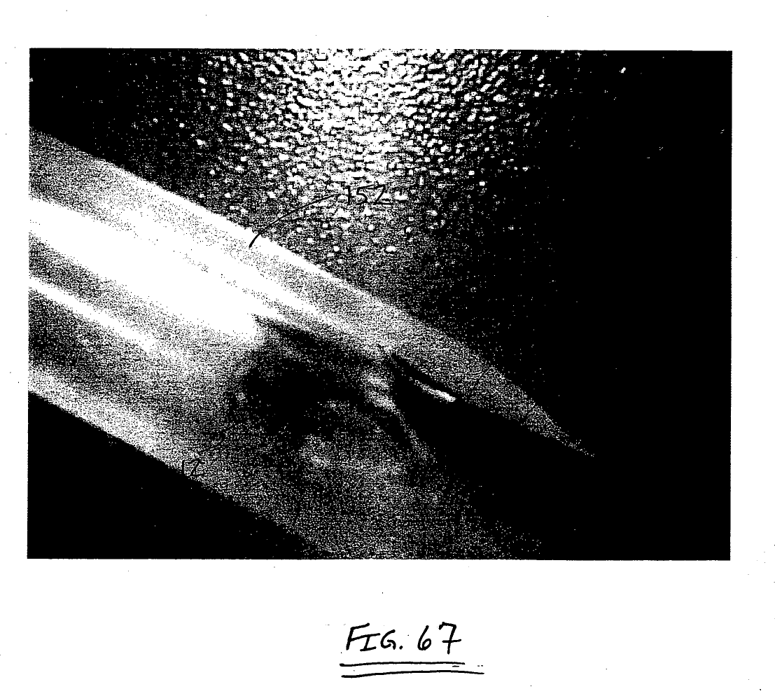

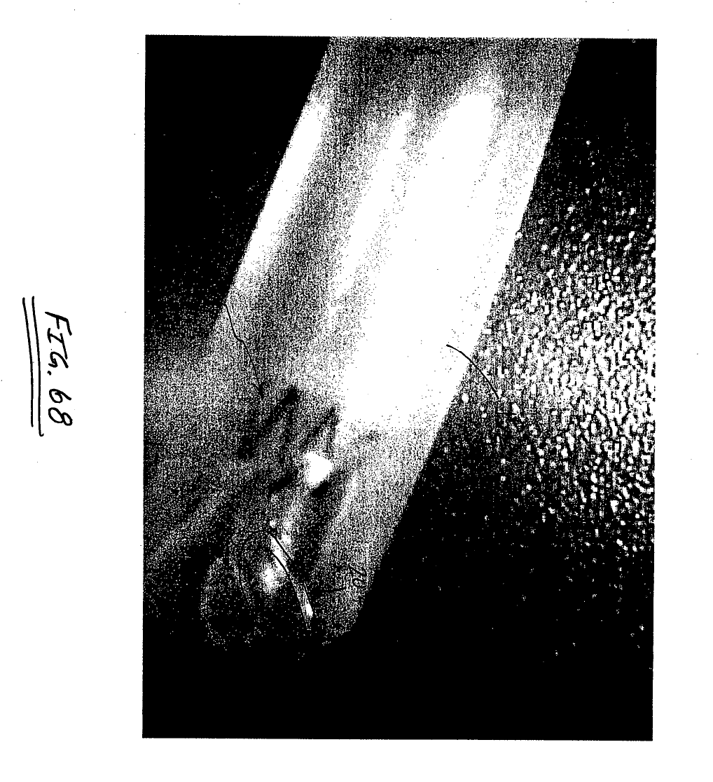

The distal structural element 12 may be secured to the distal free end of the bio-lining 14 and deployed downstream from the vascular restriction in any number of suitable fashions without departing from the scope of the present invention. One exemplary manner, by way of example only, is shown with reference to FIGS. 64-68. A needle 200 having a retractable snare 202 may be employed to encompass the distal bio-lined structural element 10 (FIGS. 64-65). At this point, the distal free end of the bio-lining 14 is wrapped over the distal structural element 12 (akin to the wrap-over described above with reference to FIGS. 56-57) and the snare 202 is sandwiching the bio-lining 14 against the exterior surface of the structural element 12. The snare 202 may then be closed (as shown in FIG. 66) such that the needle 200 may be passed through the incision and advanced to a location downstream from the vascular restriction as shown in FIG. 67. The snare 202 may then be released and the needle 200 withdrawn in order to permit the self-expanding distal structural element 12 to automatically deploy as shown in FIG. 68.

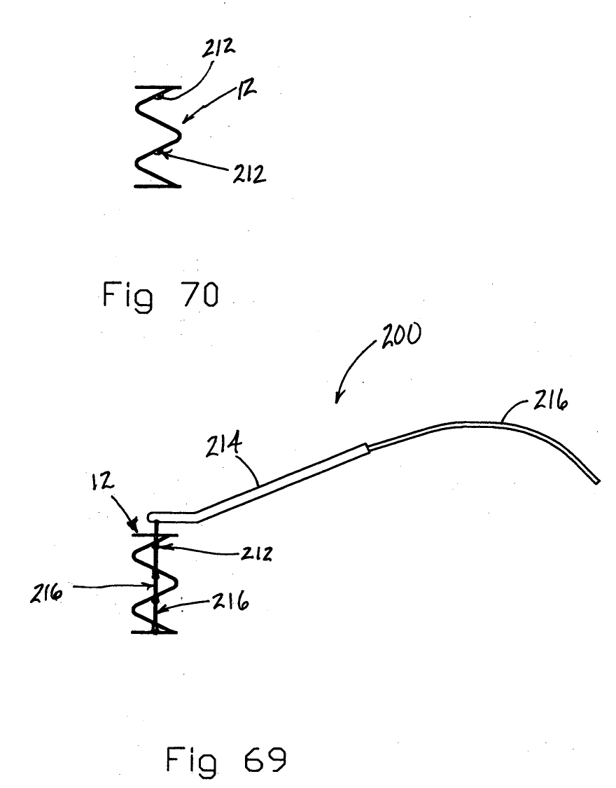

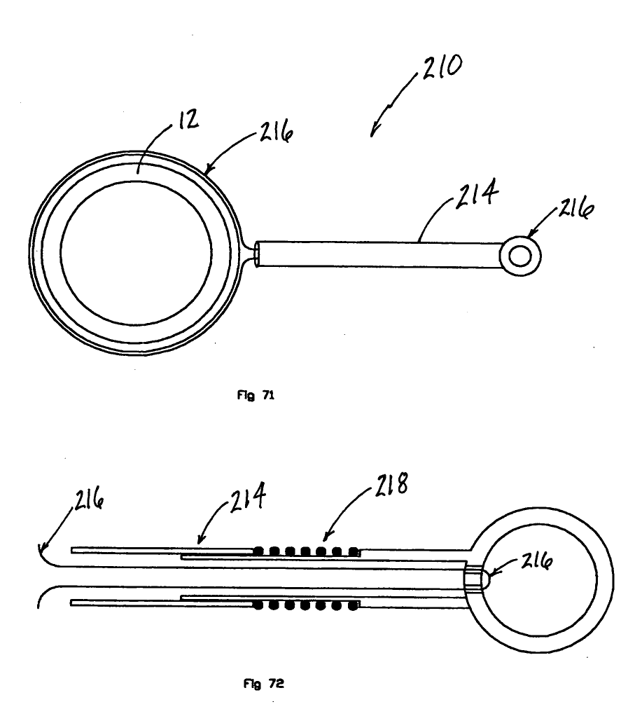

A still further manner of deploying the self-expanding structural element 12 according to the present invention will now be described with reference to FIGS. 69-72. A constricting device 210 is provided which, in conjunction with eyelets or apertures 212 formed in the self-expanding structural element 12, allows the structural element 12 to be constricted into a reduced diameter to facilitate introduction into a vascular incision 150. More specifically, the constriction device 210 includes a handle member 214 and a retractable string or thread-like element 216. The retractable thread-like element 216 is dimensioned to be advanced through the eyelets or apertures 212 (best seen in FIG. 70). The handle member 214 may be equipped to pass the thread-like element 216 therethrough such that the thread-like element 216 may be easily withdrawn to constrict (and thereby reduce the diameter of) the self-expanding structural element 12. As shown in FIG. 72, the tensioning of the thread-like element 216 may be augmented by providing the handle member 214 with a spring-loaded portion 218. Once the self-expanding structural element 12 is positioned as desired within the blood vessel, the thread-like element 216 may then be released or otherwise cut such that the structural element 12 is able to self-expand.

As evidenced by the foregoing, the various systems and methods of the present invention address the goal of overcoming vascular flow restrictions for improved blood flow. More specifically, the present invention provides systems and methods for overcoming vascular flow restrictions which involve minimizing (if not eliminating) the extent to which blood interfaces with a structural element deployed within or about a diseased vessel to restore blood flow. These inventive systems and methods accomplish this by: (1) providing at least one structural element within or about a vessel having a vascular flow restriction; and (2) equipping the structural element with bio-lining such that it restores blood flow and minimizes, if not eliminates, the interface between blood and non-biological materials.

By reducing or eliminating this “blood-device” interface, the present invention prevents (or at the very least lessens) the reformation of vascular flow restrictions within the diseased vessel.

Many alterations or modifications may be made by those of ordinary skill in the art without departing from the spirit and scope of the invention. The illustrated embodiments have been shown only for purposes of clarity and examples should not be taken as limiting the invention as defined by the following claims, which includes all equivalents, whether now or later devised.

1-38. (canceled)

39. A system for harvesting a length of autologous vessel from a patient, comprising:
an elongated element having an interior dimensioned to be advanced over a length of autologous vessel; and
a cutting element disposed at a distal end of said elongated element, said cutting element configured to extricate the exterior of said length of autologous vessel from surrounding tissue such that said extricated autologous vessel may thereafter be cut and removed from said patient.

40. The system of claim 39 and further, including an introducer dimensioned to be passed into said length of autologous vessel, wherein said elongated element is dimensioned to be advanced over said introducer to extricate the exterior of said length of autologous vessel from surrounding tissue.

41. The system of claim 40 and further, including a dilator dimensioned to be positioned within said introducer to dilate an opening formed in said autologous vessel and thereby facilitate passage of said introducer into said autologous vessel.

42. The system of claim 41 and further, including a guide-wire dimensioned to be passed through said dilator to facilitate advancement of at least one of said introducer and said dilator into said autologous vessel.

43. The system of claim 39 and further, wherein said extricated autologous vessel may be cut and removed from said patient via at least a mechanical cutting system and an electronic cutting system.

44. The system of claim 43 and further, wherein said mechanical cutting system comprises at least one of a second cutting system on said elongated element, surgical scissors, and an anvil-type cutting system comprising an anvil member capable of being introduced into said autologous vessel and advanced into abutting relation with said cutting element to sever a distal end of said autologous vessel.

45. The system of claim 44 and further, wherein said second cutting system comprises at least one cutting element hingedly coupled to said elongated element and configured to cut a distal end of said autologous vessel.

46. The system of claim 39 and further, comprising:
a system for holding said autologous vessel at least one of before, during and after said autologous vessel is extricated from said surrounding tissue, said holding system having an elongated element having a balloon capable of being selectively inflated and deflated, said balloon including a plurality of coupling members extending therefrom, wherein said balloon upon inflation will cause said coupling members to extend at least one of into and through said autologous tissue.

47. The system of claim 46 and further, wherein said holding system includes a sheath capable of protecting said interior of said autologous vessel from said coupling members on said balloon during advancement of said balloon into said autologous vessel.

48. The system of claim 39 and further, wherein said elongated element is generally cylindrical having at least one of a uniform diameter and a stepped diameter having a first diameter, a second diameter larger that said first diameter, and a tapered region extending between said first and second diameter.

49. The system of claim 48 and further, wherein said cutting element extends generally longitudinally away from said first diameter of said elongated element.

50. A method for harvesting a length of autologous vessel from a patient, comprising:
providing an elongated element having an interior dimensioned to be advanced over a length of autologous vessel, and a cutting element disposed at a distal end of said elongated element;
advancing said elongated element over a length of autologous vessel such that said cutting element extricates the exterior of said length of autologous vessel from surrounding tissue; and
removing said extricated autologous vessel from said patient.

51. The method of claim 50 and further, including providing an introducer dimensioned to be passed into said length of autologous vessel, wherein said elongated element is dimensioned to be advanced over said introducer to extricate the exterior of said length of autologous vessel from surrounding tissue.

52. The method of claim 51 and further, including providing a dilator dimensioned to be positioned within said introducer to dilate an opening formed in said autologous vessel and thereby facilitate passage of said introducer into said autologous vessel.

53. The method of claim 52 and further, including providing a guide-wire dimensioned to be passed through said dilator to facilitate advancement of at least one of said introducer and said dilator into said autologous vessel.

54. The method of claim 50 and further, wherein said step of removing may be accomplished by cutting said autologous vessel via at least a mechanical cutting system and an electronic cutting system.

55. The method of claim 54 and further, wherein said mechanical cutting system comprises at least one of a second cutting system on said elongated element, surgical scissors, and an anvil-type cutting system comprising an anvil member capable of being introduced into said autologous vessel and advanced into abutting relation with said cutting element to sever a distal end of said autologous vessel.

56. The method of claim 55 and further, wherein said second cutting system comprises at least one cutting element hingedly coupled to said elongated element and configured to cut a distal end of said autologous vessel.

57. The method of claim 50 and further, comprising the step of:
providing a system for holding said autologous vessel at least one of before, during and after said autologous vessel is extricated from said surrounding tissue, said holding system having an elongated element having a balloon capable of being selectively inflated and deflated, said balloon including a plurality of coupling members extending therefrom; and
inflating said balloon to cause said coupling members to extend at least one of into and through said autologous tissue.

58. The method of claim 57 and further, wherein said step of providing said holding system includes providing a sheath capable of protecting said interior of said autologous vessel from said coupling members on said balloon during advancement of said balloon into said autologous vessel.

59. The method of claim 50 and further, wherein said elongated element is generally cylindrical having at least one of a uniform diameter and a stepped diameter having a first diameter, a second diameter larger that said first diameter, and a tapered region extending between said first and second diameter.

60. The method of claim 59 and further, wherein said cutting element extends generally longitudinally away from said first diameter of said elongated element.

61-78. (canceled)
